SEQUENCE LISTING (35 sequences)

SEQ ID NO: 1 - DNA, 741 bp, Artificial Sequence
  Nucleic acid sequence of Hu8E5-2I scFV
caggtgcagctgcaggagagcggccccggcctgatcaagcccagccagaccctgagcctgacctgcaccgtgagcggcggcagcatcagcagcggctacaactggcactggatccggcagccccccggcaagggcctggagtggatcggctacatccactacaccggcagcaccaactacaaccccgccctgcggagccgggtgaccatcagcgtggacaccagcaagaaccagttcagcctgaagctgagcagcgtgaccgccgccgacaccgccatctactactgcgcccggatctacaacggcaacagcttcccctactggggccagggcaccaccgtgaccgtgagcagcggtggaggcggttcaggcggaggtggttctggcggtggcggatcggacatcgtgatgacccagagccccgacagcctggccgtgagcctgggcgagcgggccaccatcaactgcaagagcagccagagcctgttcaacagcggcaaccagaagaactacctgacctggtaccagcagaagcccggccagccccccaagctgctgatctactgggccagcacccgggagagcggcgtgcccgaccggttcagcggcagcggcagcggcaccgacttcaccctgaccatcagcagcctgcaggccgaggacgtggccgtgtactactgccagaacgcctacagcttcccctacaccttcggcggcggcaccaagctggagatcaagcgg

SEQ ID NO: 2 - protein, 247 aa, Artificial Sequence
  Amino acid sequence of Hu8E5-2I scFV
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKR

SEQ ID NO: 3 - DNA, 81 bp, Mus musculus
atggcctcaccgttgacccgctttctgtcgctgaacctgctgctgctgggtgagtcgattatcctggggagtggagaagct

SEQ ID NO: 4 - protein, 27 aa, Mus musculus
MASPLTRFLSLNLLLLGESIILGSGEA

SEQ ID NO: 5 - DNA, 216 bp, Artificial Sequence
  mouse CD8 hinge+Transmembrane domain
actactaccaagccagtgctgcgaactccctcacctgtgcaccctaccgggacatctcagccccagagaccagaagattgtcggccccgtggctcagtgaaggggaccggattggacttcgcctgtgatatttacatctgggcacccttggccggaatctgcgtggcccttctgctgtccttgatcatcactctcatctgctaccacaggagccga

SEQ ID NO: 6 - protein, 72 aa, Artificial Sequence
  Amino acid sequence of mouse CD8 hinge+Transmembrane domain
TTTKPVLRTPSPVHPTGTSQPQRPEDCRPRGSVKGTGLDFACDIYIWAPLAGICVALLLSLIITLICYHRSR

SEQ ID NO: 7 - DNA, 135 bp, Mus musculus
aaatggatcaggaaaaaattcccccacatattcaagcaaccatttaagaagaccactggagcagctcaagaggaagatgcttgtagctgccgatgtccacaggaagaagaaggaggaggaggaggctatgagctg

SEQ ID NO: 8 - protein, 45 aa, Mus musculus
KWIRKKFPHIFKQPFKKTTGAAQEEDACSCRCPQEEEGGGGGYEL

SEQ ID NO: 9 - DNA, 321 bp, Mus musculus
agcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcc

SEQ ID NO: 10 - protein, 107 aa, Mus musculus
SRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLA

SEQ ID NO: 11 - DNA, 66 bp, Artificial Sequence
  Nucleotide sequence of F2A
gtgaaacagactttgaattttgaccttctgaagttggcaggagacgttgagtccaaccctgggccc

SEQ ID NO: 12 - protein, 22 aa, Artificial Sequence
  Amino acid sequence of F2A
VKQTLNFDLLKLAGDVESNPGP

SEQ ID NO: 13 - DNA, 438 bp, Mus musculus
atggagaggacccttgtctgtctggtagtcatcttcttggggacagtggcccataaatcaagcccccaagggccagatcgcctcctgattagacttcgtcaccttattgacattgttgaacagctgaaaatctatgaaaatgacttggatcctgaacttctatcagctccacaagatgtaaaggggcactgtgagcatgcagcttttgcctgttttcagaaggccaaactcaagccatcaaaccctggaaacaataagacattcatcattgacctcgtggcccagctcaggaggaggctgcctgccaggaggggaggaaagaaacagaagcacatagctaaatgcccttcctgtgattcgtatgagaaaaggacacccaaagaattcctagaaagactaaaatggctccttcaaaagatgattcatcagcatctctcc

SEQ ID NO: 14 - protein, 146 aa, Mus musculus [sequence omitted]

SEQ ID NO: 15 - DNA, 57 bp, Artificial Sequence
  Nucleotide sequence of P2A
gctactaacttcagcctgctgaagcaggctggagacgtggaggagaaccctggacct

SEQ ID NO: 16 - protein, 19 aa, Artificial Sequence
  Amino acid sequence of P2A
ATNFSLLKQAGDVEENPGP

SEQ ID NO: 17 - DNA, 324 bp, Mus musculus
atggccccccgtgtgaccccactcctggccttcagcctgctggttctctggaccttcccagccccaactctggggggtgctaatgatgcggaagactgctgcctgtctgtgacccagcgccccatccctgggaacatcgtgaaagccttccgctaccttcttaatgaagatggctgcagggtgcctgctgttgtgttcaccacactaaggggctatcagctctgtgcacctccagaccagccctgggtggatcgcatcatccgaagactgaagaagtcttctgccaagaacaaaggcaacagcaccagaaggagccctgtgtct

SEQ ID NO: 18 - protein, 108 aa, Mus musculus
MAPRVTPLLAFSLLVLWTFPAPTLGGANDAEDCCLSVTQRPIPGNIVKAFRYLLNEDGCRVPAVVFTTLRGYQLCAPPDQPWVDRIIRRLKKSSAKNKGNSTRRSPVS

SEQ ID NO: 19 - DNA, 489 bp, Homo sapiens
atgagatccagtcctggcaacatggagaggattgtcatctgtctgatggtcatcttcttggggacactggtccacaaatcaagctcccaaggtcaagatcgccacatgattagaatgcgtcaacttatagatattgttgatcagctgaaaaattatgtgaatgacttggtccctgaatttctgccagctccagaagatgtagagacaaactgtgagtggtcagctttttcctgctttcagaaggcccaactaaagtcagcaaatacaggaaacaatgaaaggataatcaatgtatcaattaaaaagctgaagaggaaaccaccttccacaaatgcagggagaagacagaaacacagactaacatgcccttcatgtgattcttatgagaaaaaaccacccaaagaattcctagaaagattcaaatcacttctccaaaagatgattcatcagcatctgtcctctagaacacacggaagtgaagattcctga

SEQ ID NO: 20 - protein, 162 aa, Homo sapiens
MRSSPGNMERIVICLMVIFLGTLVHKSSSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 21 - DNA, 294 bp, Homo sapiens
atggccctgctactggccctcagcctgctggttctctggacttccccagccccaactctgagtggcaccaatgatgctgaagactgctgcctgtctgtgacccagaaacccatccctgggtacatcgtgaggaacttccactaccttctcatcaaggatggctgcagggtgcctgctgtagtgttcaccacactgaggggccgccagctctgtgcacccccagaccagccctgggtagaacgcatcatccagagactgcagaggacctcagccaagatgaagcgccgcagcagt

SEQ ID NO: 22 - protein, 98 aa, Homo sapiens
MALLLALSLLVLWTSPAPTLSGTNDAEDCCLSVTQKPIPGYIVRNFHYLLIKDGCRVPAVVFTTLRGRQLCAPPDQPWVERIIQRLQRTSAKMKRRSS

SEQ ID NO: 23 - protein, 473 aa, Artificial Sequence
  Amino acid sequence of Hu8E5-2I-28Z CAR
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 24 - protein, 471 aa, Artificial Sequence
  Amino acid sequence of Hu8E5-2I-BBZ CAR
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 25 - protein, 515 aa, Artificial Sequence
  Amino acid sequence of Hu8E5-2I-28BBZ CAR
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 26 - protein, 426 aa, Artificial Sequence
  Amino acid sequence of Hu8E5-2I-Z CAR
QVQLQESGPGLIKPSQTLSLTCTVSGGSISSGYNWHWIRQPPGKGLEWIGYIHYTGSTNYNPALRSRVTISVDTSKNQFSLKLSSVTAADTAIYYCARIYNGNSFPYWGQGTTVTVSSGGGGSGGGGSGGGGSDIVMTQSPDSLAVSLGERATINCKSSQSLFNSGNQKNYLTWYQQKPGQPPKLLIYWASTRESGVPDRFSGSGSGTDFTLTISSLQAEDVAVYYCQNAYSFPYTFGGGTKLEIKRTTTPAPRPPTPAPTIASQPLSLRPEACRPAAGGAVHTRGLDFACDIYIWAPLAGTCGVLLLSLVITRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 27 - protein, 261 aa, Artificial Sequence
  Amino acid sequence of Claudin18.1
MSTTTCQVVAFLLSILGLAGCIAATGMDMWSTQDLYDNPVTSVFQYEGLWRSCVRQSSGFTECRPYFTILGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 28 - protein, 261 aa, Artificial Sequence
  Amino acid sequence of Claudin18.2
MAVTACQGLGFVVSLIGIAGIIAATCMDQWSTQDLYNNPVTAVFNYQGLWRSCVRESSGFTECRGYFTLLGLPAMLQAVRALMIVGIVLGAIGLLVSIFALKCIRIGSMEDSAKANMTLTSGIMFIVSGLCAIAGVSVFANMLVTNFWMSTANMYTGMGGMVQTVQTRYTFGAALFVGWVAGGLTLIGGVMMCIACRGLAPEETNYKAVSYHASGHSVAYKPGGFKASTGFGSNTKNKKIYDGGARTEDEVQSYPSKHDYV

SEQ ID NO: 29 - protein, 21 aa, Homo sapiens
MALPVTALLLPLALLLHAARP

SEQ ID NO: 30 - protein, 45 aa, Homo sapiens
TTTPAPRPPTPAPTIASQPLSLRPEACRPAAGGAVHTRGLDFACD

SEQ ID NO: 31 - protein, 27 aa, Homo sapiens
FWVLVVVGGVLACYSLLVTVAFIIFWV

SEQ ID NO: 32 - protein, 41 aa, Homo sapiens
RSKRSRLLHSDYMNMTPRRPGPTRKHYQPYAPPRDFAAYRS

SEQ ID NO: 33 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 34 - protein, 42 aa, Homo sapiens
KRGRKKLLYIFKQPFMRPVQTTQEEDGCSCRFPEEEEGGCEL

SEQ ID NO: 35 - protein, 134 aa, Homo sapiens
MAQSLALSLLILVLAFGIPRTQGSDGGAQDCCLKYSQRKIPAKVVRSYRKQEPSLGCSIPAILFLPRKRSQAELCADPKELWVQQLMQHLDKTPSPQKPAQGCRKDRGASKTGKKGKGSKGCKRTERSQTPKGP

(19) United States
(12) Patent Application Publication
(10) Pub. No.: US 2022/0096544 A1
(43) Pub. Date: Mar. 31, 2022

(54) CHEMOKINE EXPRESSING CELL AND USE THEREOF

(71) Applicant: CAFA THERAPEUTICS LIMITED, Dublin (IE)

(72) Inventors: Zonghai LI, Shanghai (CN); Hong LUO, Shanghai (CN); Hua JIANG, Shanghai (CN); Huamao WANG, Shanghai (CN)

(21) Appl. No.: 17/277,142

(22) PCT Filed: Sep. 20, 2019
(86) PCT No.: PCT/CN2019/107037

§ 371 Date: Dec. 15, 2021

(30) Foreign Application Priority Data
Sep. 20, 2018 (CN) 201811102138.7
Nov. 23, 2018 (CN) 201811407486.5
Dec. 13, 2018 (CN) 201811522288.3

(51) Int. Cl. A61K 35/17; C12N 15/63; A61P 35/00; C07K 14/55; C07K 14/715; C07K 14/725; C12N 5/0783; C07K 16/30; C07K 16/28; C07K 14/705

(52) U.S. Cl. CPC A61K 35/17; C12N 15/63; A61P 35/00; C07K 14/55; C07K 14/7155; A61K 38/00; C12N 5/0637; C07K 16/30; C07K 16/2815; C07K 14/70521; C07K 14/7051

(57) ABSTRACT

The present invention relates to a chemokine expressing cell. Said cell expresses an exogenous IL-21R binding protein or an exogenous IL-21 and an exogenous chemokine. Further provided is a cell expressing an exogenous receptor, an exogenous IL-21R binding protein or an exogenous IL-21 and an exogenous chemokine. The cell involved is not only effective in solid tumor cells in vitro, but also has an excellent killing effect on solid tumor cells in vivo.

This application requires the priority of the Chinese patent application 201811102138.7 with the filing date of 2018 Sep. 20, the Chinese patent application 201811407486.5 with the filing date of 2018 Nov. 23, and the Chinese patent application 201811522288.3 with the filing date of 2018 Dec. 13. This application quotes the full text of the aforementioned Chinese patent application.

### FIELD OF THE INVENTION

The invention belongs to the field of immunotherapy. More specifically, the present invention relates to an immune effector cell that co-expresses IL-21, CCL19 and a chimeric antigen receptor.

### BACKGROUND OF THE INVENTION

In recent years, according to the discovery that the specificity of CTL's recognition of target cells depends on the T Cell Receptor (TCR), the scFv of an antibody against tumor cell-related antigens is fused with intracellular signal activation motifs such as CD3ζ or FcεRIγ of the T lymphocyte receptor to form a chimeric antigen receptor (CAR), which is genetically modified on the surface of T lymphocytes through methods such as lentiviral infection. Such CAR T lymphocytes can selectively direct T lymphocytes to tumor cells and specifically kill tumors in a manner without being limited by Major Histocompatibility Complex (MHC).

The chimeric antigen receptor comprises an extracellular binding domain, a transmembrane domain and an intracellular signaling domain. Usually the extracellular domain comprises an scFv that can recognize tumor-associated antigens, a transmembrane domain derived from the transmembrane region of a molecule such as CD8, CD28, and an intracellular signaling domain derived from the immunoreceptor tyrosine activation motif (ITAM) CD3ζ or FcεRIγ and a costimulatory signal molecule derived from the intracellular signaling domain of CD28, CD27, CD137 or CD134, etc.

However, due to the complexity of the microenvironment of organisms, especially solid tumors, drug candidates that show excellent effects in vitro often fail to show corresponding effects in vivo. In other words, the in vitro results of the drug candidates cannot reasonably predict the effect in vivo. In addition, the same antibody has different effects on different tumors with the same target site. For example, Trastuzumab has a good therapeutic effect when applied to HER2-positive breast cancers, while has no effect when applied to HER2-positive gastric cancer (Fu Qiang, etc. Progress of HER2 signaling pathway in gastric cancer and the clinical application of Trastuzumab, Drug Evaluation, 2012, 9(27): 8-12).

Although immune effector cells have attractive prospects in tumor immunotherapy, their efficacy in solid tumors is still not significant. The survival rate of immune effector cells in tumor tissues is relatively poor and the activity is not high.

Therefore, further research is still needed in this field to further improve the immunotherapeutic efficacy of immune effector cells to tumors, especially to develop effective immune effector cells for solid tumors.

### SUMMARY OF THE INVENTION

The purpose of the present invention is to provide an immune effector cell with improved therapeutic effect on tumor immunotherapy, especially an immune effector cell with an effective killing effect on solid tumors.

In the first aspect, the present invention provides a chemokine-expressing cell, wherein the cell expresses an exogenous IL-21R binding protein or an exogenous IL-21, and an exogenous chemokine; preferably, the chemokine is an exogenous CCL19 or CCL21.

In a specific embodiment, the cell expresses an exogenous IL-21 and an exogenous chemokine; preferably, the chemokine is an exogenous CCL19 or CCL21; more preferably, the chemokine is CCL19.

In a specific embodiment, the exogenous IL-21 is a wild-type IL-21 or a variant or truncated fragment of the wild-type IL-21, the variant or truncated fragment has the same or similar function as the wild-type IL-21;

In a specific embodiment, the exogenous IL-21 is a human or murine IL-21;

In a specific embodiment, the amino acid sequence of the exogenous IL-21 has at least 90% identity with the sequence shown in SEQ ID NO: 20.

In the present invention, the exogenous IL-21R binding protein or the exogenous IL-21 can be expressed constitutively or inductively.

In a specific embodiment, the exogenous IL-21R binding protein or the exogenous IL-21 is expressed inductively. Preferably, the promoter for expressing the exogenous IL-21R binding protein or the exogenous IL-21 is an inducible promoter of an immune cell; preferably, the inducible promoter of an immune cell is the NFAT6 promoter.

In a specific embodiment, the exogenous IL-21R binding protein can specifically bind to IL-21R and enhance IL-21R activity.

In a specific embodiment, the IL-21R binding protein is selected from IL-21R antibodies.

In a specific embodiment, the exogenous CCL19 is a wild-type CCL19 or a variant or truncated fragment of the wild-type CCL19, and the variant or truncated fragment has the same or similar function as the wild-type CCL19.

In a specific embodiment, the CCL19 is a human or murine CCL19.

In a specific embodiment, the amino acid sequence of the CCL19 has at least 90% identity with the sequence shown in SEQ ID NO: 22.

In a specific embodiment, the exogenous CCL21 is a wild-type CCL21 or a variant or truncated fragment of the wild-type CCL21, and the variant or truncated fragment has the same or similar function as the wild-type CCL21.

In the present invention, the CCL21 can be a human or murine CCL21.

In a specific embodiment, the CCL21 is a human CCL21.

In a specific embodiment, the amino acid sequence of the CCL21 has at least 90% identity with the sequence shown in SEQ ID NO: 35.

In the present invention, the exogenous chemokine can be expressed constitutively or inductively.

In a specific embodiment, the exogenous chemokine is expressed inductively. Preferably, the promoter used to express the chemokine is an inducible promoter of an immune cell; preferably, the inducible promoter of an immune cell is an NFAT6 promoter.

In a specific embodiment, the cell is an immune effector cell.

In a specific embodiment, the immune effector cell is selected from the group consisting of: a T cell, a B cell, a natural killer (NK) cell, and a natural killer T (NKT) cell, a mast cell, or a bone marrow-derived phagocyte, or a combination of at least two of them. In a preferred embodiment, the immune effector cell is a T cell, a B cell, or a NKT cell.

In a specific embodiment, the cell is derived from an autologous cell or an allogeneic cell. Preferably, the cell is an autologous T cell, an allogeneic T cell, or an allogeneic NK cell; more preferably, the T cell is an autologous T cell.

In a specific embodiment, the cell further expresses an exogenous receptor that specifically binds to a target antigen.

In a specific embodiment, the target antigen is a tumor antigen.

In a specific embodiment, the tumor antigen is selected from the group consisting of: thyroid stimulating hormone receptor (TSHR), CD171, CS-1, C-type lectin-like molecule-1, ganglioside GD3, Tn antigen, CD19, CD20, CD22, CD30, CD70, CD123, CD138, CD33, CD44, CD44v7/8, CD38, CD44v6, B7H3 (CD276), B7H6, KIT (CD117), interleukin 13 receptor subunit α (IL-13Rα), interleukin 11 receptor α (IL-11Rα), prostate stem cell antigen (PSCA), prostate specific membrane antigen (PSMA), carcinoembryonic antigen (CEA), NY-ESO-1, HIV-1 Gag, MART-1, gp100, tyrosinase, mesothelin, EpCAM, protease serine 21 (PRSS21), vascular endothelial growth factor receptor, Lewis (Y) antigen, CD24, platelet-derived growth factor receptor β (PDGFR-β), stage-specific embryonic antigen-4 (SSEA-4), cell surface-associated mucin 1 (MUC1), MUC6, epidermal growth factor 20 receptor family and the mutants thereof (EGFR, EGFR2, ERBB3, ERBB4, EGFRvIII), nerve cell adhesion molecule (NCAM), carbonic anhydrase IX (CAIX), LMP2, ephrin A receptor 2 (EphA2), fucosyl GM1, sialyl Lewis adhesion molecule (sLe), ganglioside, TGS5, high molecular weight melanoma-associated antigen (HMWMAA), o-acetyl GD2 ganglioside (OAcGD2), folate receptor, tumor vascular endothelial marker 25 1 (TEM1/CD248), tumor vascular endothelium marker 7 related (TEM7R), Claudin6, Claudin18.2 (CLD18A2), Claudin18.1, ASGPR1, CDH16, 5T4, 8H9, αvβ6 integrin, B cell maturation antigen (BCMA), CA9, kappa light chain (κ light chain), CSPG4, EGP2, EGP40, FAP, FAR, FBP, embryonic AchR, HLA-A1, HLA-A2, MAGEA1, MAGE3, KDR, MCSP, NKG2D ligand, PSC1, ROR1, Sp17, SURVIVIN, TAG72, TEM1, fibronectin, tenascin, carcinoembryonic variants of tumor necrosis zone, G protein-coupled receptor C group 5-member D (GPRCSD), X chromosome open reading frame 61 (CXORF61), CD97, CD179a, anaplastic lymphoma kinase (ALK), polysialic acid, placenta specific 1 (PLAC1), globoH glycoceramide Hexose part (GloboH), breast differentiation antigen (NY-BR-1), uroplakin 2 (UPK2), hepatitis A virus cell receptor 1 (HAVCR1), adrenaline receptor 5β3 (ADRB3), pannexin 3 (PANX3), G protein coupled receptor 20 (GPR20), lymphocyte antigen 6 complex locus K9 (LY6K), olfactory receptor 51E2 (OR51E2), TCRγ alternating reading frame protein (TARP), Wilms tumor protein (WT1), ETS translocation variant gene 6 (ETV6-AML), sperm protein 17 (SPA17), X antigen family member 1A (XAGE1), angiogenin-binding cell surface receptor 2 (Tie2), melanoma cancer testis antigen-1 (MAD-CT-1), melanoma cancer testis antigen-2 (MAD-CT-2), Fos-related antigen 1, mutant p53-10, human telomerase reverse transcriptase (hTERT), sarcoma translocation breakpoint, melanoma inhibitor of apoptosis (ML-IAP), ERG (transmembrane protease serine 2 (TMPRSS2) ETS fusion gene), N-acetylglucosaminyl transferase V (NA17), paired box protein Pax-3 (PAX3), androgen receptor, cyclin B1, V-myc avian myelocytomatosis viral related oncogene, neuroblastoma derived homolog (MYCN), Ras homolog family member C (RhoC), cytochrome P450 1B1 (CYP1B1), CCCTC binding factor (zinc finger protein)-like (BORIS), Squamous cell carcinoma antigen recognized by T-cells 3 (SART3), paired box protein Pax-5 (PAX5), proacrosin binding protein sp32 (OYTES1), lymphocyte-specific protein tyrosine kinase (LCK), A kinase anchoring protein 4 (AKAP-4), synovial sarcoma X breakpoint 2 (SSX2), CD79a, CD79b, CD72, leukocyte-related immunoglobulin-like receptor 1 (LAIR1), Fc fragment of IgA receptor (FCAR), leukocyte immunoglobulin-like receptor subfamily member 2 (LILRA2), CD300 molecular-like family member f (CD300LF), C-type lectin domain family 12 member A (CLEC12A), bone marrow stromal cell antigen 2 (BST2), EGF-like module containing, mucin-like, hormone receptor-like 2 (EMR2), lymphocyte antigen 75 (LY75), glypican-3 (GPC3), Fc receptor-like 5 (FCRL5), immunoglobulin λ-like polypeptide 1 (IGLL1).

In a specific embodiment, the target antigen is a pathogen antigen

In a specific embodiment, the pathogen antigen is selected from the group consisting of: virus, bacteria, fungus, protozoa, or parasite antigen; in one embodiment, the viral antigen is selected from the group consisting of: cytomegaloviral antigen, Epstein-Barr viral antigen, human immunodeficiency viral antigen or influenza viral antigen.

In a specific embodiment, the target antigen is a hematologic tumor-associated antigen.

In a specific embodiment, the hematologic tumor-associated antigen is selected from the group consisting of: CD19, CD20, BCMA and CD30.

In a specific embodiment, the target antigen is a solid tumor-associated antigen.

In a specific embodiment, the solid tumor-related antigen is selected from the group consisting of: prostate specific membrane antigen, carcinoembryonic antigen, IL13Ralpha, HER-2, NY-ESO-1, Lewis Y, MART-1, gp100, tyrosinase, WT-1, hTERT, mesothelin, EGFR, EGFRvIII, glypican 3 (GPC3), EphA2, HER3, EpCAM, MUC1, MUC16, claudin 18.2 (CLD18A2), claudin 18.1 (CLD18A1), folate receptor, claudin 6, CD138, MAGE3, ASGPR1 and CDH16. Preferably, the solid tumor-associated antigen is selected from the group consisting of: mesothelin, EGFR, EGFRvIII, glypican 3, claudin 18.2, claudin 6 and IL13Ralpha; more preferably, the solid tumor-associated antigen is claudin 18.2.

In a specific embodiment, the exogenous receptor has an antigen-binding domain, a transmembrane domain, and an intracellular domain, and the antigen-binding domain specifically binds to the target antigen.

In a specific embodiment, the exogenous receptor is selected from the group consisting of: a chimeric antigen receptor (CAR), a modified T cell (antigen) receptor (TCR), a T cell fusion protein (TFP), a T cell antigen coupler (TAC), or a combination thereof.

In a specific embodiment, the exogenous receptor is a chimeric antigen receptor

In a specific embodiment, the antigen binding domain of the chimeric antigen receptor comprises: an antibody, an antibody fragment, an scFv, an Fv, a Fab, a (Fab′)2, a single domain antibody (SDAB), a VH or VL domain, or a camelid VHH domain, or a natural ligand of the corresponding antigen, or a combination thereof.

In a specific embodiment, the transmembrane domain of the chimeric antigen receptor comprises a transmembrane domain of a protein selected from the group consisting of: α, β or ζ chain of T cell receptor, CD28, CD3ε, CD45, CD4, CD5, CD8, CD9, CD16, CD22, CD33, CD37, CD64, CD80, CD86, CD134, CD137, CD154, KIRDS2, OX40, CD2, CD27, LFA-1(CD11a, CD18), ICOS(CD278), 4-1BB(CD137), GITR, CD40, BAFFR, HVEM(LIGHTR), SLAMF7, NKp80(KLRF1), CD160, CD19, IL2Rβ, IL2Rγ, IL7Rα, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1(CD226), SLAMF4(CD244, 2B4), CD84, CD96(Tactile), CEACAM1, CRTAM, Ly9(CD229), CD160(BY55), PSGL1, CD100(SEMA4D), SLAMF6(NTB-A, Ly108), SLAM(SLAMF1, CD150, IPO-3), BLAME(SLAMF8), SELPLG(CD162), LTBR, PAG/Cbp, NKp44, NKp30, NKp46, NKG2D and NKG2C.

In a specific embodiment, the intracellular domain of the chimeric antigen receptor comprises: a primary signaling domain and/or a costimulatory signaling domain, wherein:

(1) the primary signaling domain comprises a functional signaling domain of a protein selected from the group consisting of: CD3ζ, CD3γ, CD3δ, CD3ε, common FcRγ (FCER1G), Fc (FcεR1b), CD79a, CD79b, FcγRIIa, DAP10 and DAP12, or a combination thereof; and/or

(2) the costimulatory signaling domain comprises a functional signaling domain of a protein selected from the group consisting of: CD27, CD28, 4-1BB (CD137), OX40, CD30, CD40, PD-1, ICOS, lymphocyte function related antigen-1 (LFA-1), CD2, CD7, LIGHT, NKG2C, B7-H3, ligands that specifically bind to CD83, CDS, ICAM-1, GITR, BAFFR, HVEM (LIGHTR), SLAMF7, NKp80 (KLRF1), CD160, CD19, CD4, CD8α, CD8β, IL2Rβ, IL2Rγ, IL7Rα, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, TRANCE/RANKL, DNAM1(CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, NKp44, NKp30, NKp46 and NKG2D, or a combination thereof.

In a specific embodiment, the chimeric antigen receptor comprises:

(i) an antibody or fragment thereof that specifically binds to an antigen, the transmembrane domain of CD28 or CD8, the costimulatory signaling domain of CD28, and CD3′; or

(ii) an antibody or fragment thereof that specifically binds to an antigen, the transmembrane domain of CD28 or CD8, the costimulatory signaling domain of CD137, and CD3ζ; or

(iii) an antibody or fragment thereof that specifically binds to an antigen, the transmembrane domain of CD28 or CD8, the costimulatory signaling domain of CD28, the costimulatory signaling domain of CD137, and CD3ζ.

In a specific embodiment, the amino acid sequence of the antigen binding domain comprises a sequence that has at least 90% identity with the sequence shown in SEQ ID NO: 2.

In a specific embodiment, the amino acid sequence of the exogenous receptor has at least 90% identity with the sequence shown in SEQ ID NO: 23, 24, 25, and 26.

In a specific embodiment, the exogenous receptor, and/or the exogenous IL-21R binding protein or exogenous IL-21, and/or the chemokine are expressed using a viral vector; preferably, the viral vector includes: a lentiviral vector, a retroviral vector or an adenoviral vector.

In a specific embodiment, the expression of an inhibitory immune checkpoint in the cell is down-regulated.

In a specific embodiment, the expression of PD-1, LAG-3 and/or TIM-3 is down-regulated in the cells.

In the second aspect of the present invention, provided is an expression construct, wherein the expression construct comprises the following that are sequentially connected: an expression cassette 1 of an exogenous receptor that specifically binds to a target antigen, an expression cassette 2 of an exogenous IL-21R binding protein or an exogenous IL-21, and an expression cassette 3 of a chemokine; preferably, the expression cassettes are connected by tandem fragments, selected from the group consisting of F2A, P2A, T2A, and/or E2A.

In the third aspect of the present invention, provided is an expression vector, comprising the expression construct described in the second aspect.

In the fourth aspect of the present invention, provided is a virus, comprising the expression vector described in the third aspect.

In the fifth aspect of the present invention, provided is a method for improving the viability of immune response cells, wherein the method comprises the co-expression of the following in immune response cells: a chimeric antigen receptor that specifically binds to a target antigen, an exogenous IL-21R binding protein or an exogenous IL-21, and an exogenous chemokine.

In a specific embodiment, the chemokine is CCL19.

In the sixth aspect of the present invention, provided is the use of the cell described in the first aspect of the present invention, or the expression construct described in the second aspect of the present invention, or the expression vector described in the third aspect of the present invention, or the virus in the fourth aspect of the present invention, for preparing pharmaceutical compositions for inhibiting tumors, inhibiting pathogens or strengthening subjects' immune tolerance.

In a specific embodiment, the tumors include: breast cancer, blood cancer, colon cancer, rectal cancer, renal cell carcinoma, liver cancer, non-small cell carcinoma of the lung, small intestine cancer, esophagus cancer, melanoma, bone cancer, pancreatic cancer, skin cancer, head and neck cancer, skin or intraocular melanoma, uterine cancer, ovarian cancer, rectal cancer, anal cancer, stomach cancer, testicular cancer, uterine cancer, fallopian tube cancer, endometrial cancer, cervical cancer, vaginal cancer, vulvar cancer, Hodgkin's disease, non-Hodgkin's lymphoma, endocrine system cancer, thyroid cancer, parathyroid cancer, adrenal cancer, soft tissue sarcoma, urethral cancer, penis cancer, pediatric solid tumor, bladder cancer, renal or ureteral cancer, renal pelvic cancer, central nervous system (CNS) tumor, primary CNS lymphoma, tumor angiogenesis, spinal tumor, glioma, pituitary adenoma, Kaposi's sarcoma, epidermoid carcinoma, squamous cell carcinoma, T-cell lymphoma, environmentally induced cancers, or combinations and metastasis thereof.

In the seventh aspect of the present invention, provided is a pharmaceutical composition, comprising: the cell described in the first aspect and a pharmaceutically acceptable carrier or excipient.

In the eighth aspect of the present invention, provided is a kit, wherein the kit comprises: the cell described in the first aspect or the pharmaceutical composition described in the seventh aspect.

In the ninth aspect of the present invention, provided is a medicine box, comprising medicine box A and medicine box B, the medicine box A comprises a chemokine-expressing cell, and the medicine box B comprises an exogenous IL-21R binding protein or an exogenous IL-21;

or, the medicine box A comprises an exogenous IL-21R binding protein or exogenous IL-21 expressing-cell, and the medicine box B comprises an exogenous chemokine;

or, the medicine box A comprises an immune effector cell, and the medicine box B comprises an exogenous IL-21R binding protein or an exogenous IL-21, and an exogenous chemokine;

preferably, the chemokine is an exogenous CCL19 or CCL21.

In the medicine box as described above, wherein:

further preferred definitions of the exogenous IL-21, the exogenous IL-21R binding protein, the exogenous CCL19, the exogenous CCL21, and the immune effector cells are as described in the first aspect of the present invention.

### The Beneficial Effect of the Present Invention

The chemokine-expressing cell provided in the present invention can be used to produce CAR-T cells with survival ability, lymphocyte accumulation ability, tumor cell damage activity, and CAR-T cells that are resistant to immunosuppression in the cancer microenvironment, and to increase their therapeutic effect on solid tumors. By using the above CAR-T cells to implement immunotherapy for cancer patients, it is possible to obtain cancer immunotherapy that is expected to have a strong therapeutic effect for cancer and is still effective for refractory and progressive cancers.

### BRIEF DESCRIPTION OF THE DRAWINGS

FIG. 1A is the plasmid map of MSCV-hu8E5 (2I)-mBBZ; FIG. 1B is the plasmid map of MSCV-hu8E5 (2I)-mBBZ-F2A-mIL-21-P2A-mCCL19.

FIG. 2 shows the cytokine secretion of mBBZ CART cells and mBBZ-21*19 CAR T.

FIG. 3A and FIG. 3B show the results of PD-1 secretion.

FIG. 4A and FIG. 4B show the results of LAG3 secretion.

FIG. 5A and FIG. 5B show the results of TIM-3 secretion.

FIG. 6 shows the results of the in vitro killing toxicity test.

FIG. 7 shows the anti-tumor effects against subcutaneous transplanted tumors.

### DETAIL DESCRIPTION OF THE INVENTION

After extensive and in-depth research, the inventor unexpectedly discovered that immune effector cells expressing chimeric antigen receptors, IL-21 and CCL19 are not only effective against solid tumor cells in vitro, but also have a better killing effect on solid tumor cells in vivo, thereby improving the survival and function of immune effector cells in tumors. The present invention has been completed on this basis.

The Term

Unless specifically defined, all technical and scientific terms used herein have the same meanings commonly understood by those skilled in the fields of gene therapy, biochemistry, genetics, and molecular biology. All methods and materials similar or equivalent to those described herein can be used in the practice or testing of the present invention, wherein suitable methods and materials are described herein. All publications, patent applications, patents and other references mentioned herein are incorporated herein by reference in their entirety. In case of conflict, the specification, including definitions, will control. In addition, unless otherwise specified, the materials, methods, and examples are illustrative only and not intended to be limiting.

Unless otherwise specified, the practice of the present invention will use traditional techniques of cell biology, cell culture, molecular biology, transgenic biology, microbiology, recombinant DNA and immunology, which all fall within the technical scope of the art. These techniques are fully explained in the literature. See, for example, Current Protocols in Molecular Biology (Frederick M. AUSUBEL, 2000, Wiley and son Inc, Library of Congress, USA); Molecular Cloning: A Laboratory Manual, Third Edition, (Sambrook et al., 2001, Cold Spring Harbor, New York: Cold Spring Harbor Laboratory Press); Oligonucleotide Synthesis (M. J. Gaited., 1984); Mullis et al. U.S. Pat. No. 4,683,195; Nucleic Acid Hybridization (B. D. Harries & S. J. Higgins eds. 1984); Transcription And Translation (B. D. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), especially Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Hand book Of Experimental Immunology, vol. I-IV (D. M. Weir and C. C. Blackwell, eds., 1986); and Manipulating the Mouse Embryo (Cold Spring Harbor Laboratory Press, Cold Spring Harbor, N.Y., 1986).

The term “immune effector cell” refers to a cell that participates in an immune response, for example, that promotes an immune effect. Examples of immune effector cells include T cells (for example, α/β T cells and γ/δ T cells), B cells, natural killer (NK) cells, natural killer T (NKT) cells, mast cells, and bone marrow-derived phagocytes. Preferably, the T cells include autologous T cells, xenogeneic T cells, and allogeneic T cells, and the natural killer cells are allogeneic NK cells. As used herein, the term “immune effector function or immune effector response” refers to, for example, the function or response of immune effector cells for enhancing or promoting the immune attack to target cells. For example, immune effector function or response refers to the properties of T cells or NK cells that promote the killing or the inhibition of growth or proliferation of target cells.

“Interleukin 21 (IL-21 or IL21)” is a type I cytokine discovered by Parrish Novak et al. in 2000. It is produced by activated CD4+ T cells, NKT cells, Tfh cells and Th17 cells, and has high homology with IL-2, IL-4, IL-15, belonging to the γc family member. hIL-21 (human IL-21) is located on the long arm of chromosome 4 (4q26 27) and transcribes a mature mRNA consisting of 642 nucleotides which encodes a protein precursor consisting of 162 amino acids, wherein the first 31 amino acids are the signal peptide, the following 131 amino acids constitute a mature IL-21 with a four-helix domain and a molecular weight of 15 KD. The 5′ regulatory region of IL-21 comprises three binding sites of nuclear factor of activated T cells (NF-AT). And the activity of the IL-21 promoter is produced by the action of calcium ionophores on cells. IL-21 has two DNaseI hypersensitive sites, both of which are conserved in humans and mice. One of them is located in the IL-21 promoter region and is related to TCR-mediated IL-21 transcription. hIL-21 can specifically bind to human interleukin 21 receptor (hIL-21R), activating JAK/STAT and other signaling pathways, and exhibit complex biological effects. It can regulate the differentiation, apoptosis and antibody subclass-production of B cells, promote T cell-mediated acquired immunity, enhance the cytotoxicity and IFN γ-producing ability of NK cells, and mediate the transition between active immunity and passive immunity. rhiL 21 plays an important role in allergic reaction, inflammatory reaction, autoimmune reaction and clinical applications such as anti-tumor application.

In the present invention, the term “IL-21” refers to a protein (preferably derived from mammals, such as mice or humans) that can interact (for example, bind) with IL-21R (NM 021798.3, SEQ ID NO: 14)) (preferably from a mammal, such as, murine or human IL-21), and has one of the following characteristics: (i) an amino acid sequence of a naturally occurring mammalian IL-21 or a fragment thereof, such as the amino acid sequence shown in SEQ ID NO: 20 (human) or a fragment thereof; (ii) an amino acid sequence that substantially has, for example, at least 85%, 90%, 95%, 98%, 99% homology with the amino acid sequence shown in SEQ ID NO: 20 (human) or a fragment thereof; (iii) an amino acid sequence encoded by a naturally-occurring mammalian IL-21 nucleotide sequence or a fragment thereof (such as SEQ ID NO: 19 (human) or a fragment thereof); (iv) an amino acid sequence encoded by a nucleotide sequence having, for example, at least 85%, 90%, 95%, 98%, 99% homology with the nucleotide sequence shown in SEQ ID NO: 19 (human) or a fragment thereof; (v) an amino acid sequence encoded by a nucleotide sequence that is degenerate from the naturally occurring IL-21 nucleotide sequence or a fragment thereof (for example, SEQ ID NO: 19 (human) or a fragment thereof); or (vi) a nucleotide sequence that hybridizes to one of the aforementioned nucleotide sequences under strict conditions, such as highly strict conditions.

“Enhancing IL-21R activity” should be understood to mean any way that can enhance the IL-21R signaling pathway, including the modification of IL-21 receptors, and the IL-21R binding protein of the present disclosure, enhancing any one or more of the activities of the naturally occurring IL-21R to activate the downstream signal molecules of the pathway, including but not limited to stimulating the proliferation, cytotoxicity or maturation of NK cells; stimulating the proliferation or differentiation of B cells and T cells; stimulating the production and affinity maturation of antibodies in B cells; stimulating the cytotoxicity of CD8+ T cells; stimulating the production of interferon γ in T cells and NK cells; inhibiting the activation and maturation of dendritic cells (DC); inhibiting the release of inflammatory mediators from mast cells; enhancing phagocytosis of macrophages; inhibiting the generation or survival of TReg cells; and stimulating the proliferation of bone marrow progenitor cells. Exogenous IL-21R binding protein refers to all proteins that can specifically bind to IL-21R and enhance the activity of IL-21R.

In the present invention, the term “CCL19 (Chemokine (C-C motif) ligand 19, CCL19)” belongs to CC chemokines, also known as EBV-induced molecule 1 ligand chemokine (ELC) or human macrophage inflammatory protein 3B (MIP-3B), the coding gene of which is located on the short arm of chromosome 9. It is mainly expressed in T cells in secondary lymphoid tissues and organs such as spleen and lymph nodes, making naive T cells and mature DC cells chemotactic. CCL19 can induce T cells, DC cells and NK cells in anti-tumor activities, such as cytotoxicity, antigen presentation, phagocytosis and cytokine secretion, to inhibit tumor proliferation, migration and invasion. In the present invention, CCL19 has one of the following features: (i) an amino acid sequence of naturally occurring mammalian CCL19 or a fragment thereof, such as the amino acid sequence shown in SEQ ID NO: 22 (human) or a fragment thereof; (ii) an amino acid sequence that substantially has, for example, at least 85%, 90%, 95%, 98%, 99% homology with the amino acid sequence shown in SEQ ID NO: 22 (human) or a fragment thereof; (iii) an amino acid sequence encoded by the naturally-occurring mammalian CCL19 nucleotide sequence or a fragment thereof (such as SEQ ID NO: 21 (human) or a fragment thereof); (iv) an amino acid sequence encoded by a nucleotide sequence having, for example, at least 85%, 90%, 95%, 98%, 99% homology with the nucleotide sequence shown in SEQ ID NO: 21 (human) or a fragment thereof; (v) an amino acid sequence encoded by a nucleotide sequence that is degenerate from the naturally occurring CCL19 nucleotide sequence or a fragment thereof (for example, SEQ ID NO: 21 (human) or a fragment thereof); or (vi) a nucleotide sequence that hybridizes to one of the aforementioned nucleotide sequences under strict conditions, such as highly strict conditions.

In the present invention, CCL21 belongs to the CC chemokines and is mainly expressed in peripheral lymph tissues. Because of the unique structure of 6 consecutive cysteine sequences and the wide expression in secondary lymphoid tissues, it is also called 6Ckine or secondary lymphoid tissue chemokine. CCL21 has a chemotactic effect on a variety of immune effector cells, so it plays an important role in tumors, autoimmune diseases, acquired immunodeficiency syndrome and other diseases. CCL21 in the present invention has one of the following features: (i) an amino acid sequence of naturally occurring mammalian CCL21 or a fragment thereof, such as the amino acid sequence shown in SEQ ID NO: 35 (human) or a fragment thereof; (ii) an amino acid sequence that substantially has, for example, at least 85%, 90%, 95%, 98%, 99% homology with the amino acid sequence shown in SEQ ID NO: 35 (human) or a fragment thereof.

The terms “therapeutically effective amount”, “therapeutically effective”, “effective amount” or “in an effective amount” are used interchangeably herein and refer to an amount of a compound, preparation, substance or composition that effectively achieves a specific biological result as described herein, such as but not limited to an amount or dose sufficient to promote T cell response. When indicating “immunologically effective amount”, “anti-tumor effective amount”, “tumor-suppressing effective amount” or “therapeutically effective amount”, the precise number of immune effector cells and therapeutic agents of the present invention to be administered can be determined by the physician in consideration of the individual's age, weight, size of tumors, degree of infection or metastasis, and the condition of the patient (subject). An effective amount of immune effector cells refers to, but is not limited to, the number of immune effector cells that can: increase, enhance or prolong the anti-tumor activity of immune effector cells; increase the number of anti-tumor immune effector cells or activated immune effector cells; promote IFN-γ secretion; cause tumor regression, tumor shrinkage, and tumor necrosis.

The term “T cell (antigen) receptor (TCR)” is a characteristic marker on the surface of all T cells, which binds to CD3 by a non-covalent bond to form a TCR-CD3 complex. TCR is responsible for recognizing antigens bound to major histocompatibility complex molecules. TCR is a heterodimer composed of two different peptide chains, composed of two peptide chains α and β. Each peptide chain can be divided into several parts such as variable region (V region), constant region (C region), transmembrane region and cytoplasmic region; its characteristic is that the cytoplasmic region is very short. TCR molecules belong to the immunoglobulin superfamily, and their antigen specificity exists in the V regions. Each of the V regions (Vα, Vβ) has three hypervariable regions CDR1, CDR2, and CDR3, wherein CDR3 has the largest amount of variation, which directly determines the antigen binding specificity of TCR. When TCR recognizes the MHC-antigen peptide complex, CDR1 and CDR2 recognize and bind to the side wall of the antigen binding groove of the MHC molecule, and CDR3 directly binds to the antigen peptide. TCRs are divided into two categories: TCR1 and TCR2. TCR1 is composed of two chains, γ and δ, and TCR2 is composed of two chains, α and β. The recognition ability of these natural (or manufactured by other means) “anti-cancer” T cells is often weak, so they cannot form a favorable attack on cancer cells. In this case, the “affinity” and combat effectiveness of these TCRs to the corresponding TAA can be improved through the modification of a part of genes, that is, high-affinity TCR. “Gene modified TCR” technology is therefore called “Affinity-Enhanced TCR” technology. The gene modified T cell receptor (Gene Modified TCR) is used to form a chimeric TCR molecule (chim-TCR) by using the constant region domains of the heavy and light chains of the antibody that belong to the same immunoglobulin superfamily with the TCR molecule to replace the constant region domains of its β chain and a chain, respectively.

CD3 (Cluster of Differentiation 3), a T cell co-receptor, is a protein complex composed of four different chains. In mammals, the complex comprises one CD3γ chain, CD3δ chain, and two CD3ε chains. These chains have a molecule called the accessory T cell receptor (TCR) and a zeta-chain to generate activation signals for T lymphocytes. The TCR, ζ chain and CD3 molecule together constitute a T cell receptor complex. The CD3 molecule is connected to T cell receptor (TCR) through a salt bridge to form a TCR-CD3 complex, which participates in the signaling of T cells, and is mainly used to label thymocytes, T lymphocytes and T cell lymphomas. The cytoplasmic segment of CD3 contains immunoreceptor tyrosine-based activation motif (ITAM). TCR recognizes and binds the antigen peptide presented by the MHC (major histo-compatibility complex) molecule, resulting in that the tyrosine residues of the conserved sequence in the ITAM of CD3 are phosphorylated by the tyrosine protein kinase p56lck in T cells, and then other tyrosine protein kinases containing SH2 (Scr homology 2) domain (such as ZAP-70) can be recruited. The phosphorylation of ITAM and the binding of ZAP-70 are one of the important biochemical reactions in the early stages of the signaling process of T cell activation. Therefore, the function of the CD3 molecule is to transduce the activation signal generated by the recognition of the antigen by TCR. In this application, the exogenous receptor that can bind to the target antigen and can trigger the activation of CD3 signals comprises at least one CD3 binding site and at least an additional antigen binding site specific for bacterial substances, viral proteins, autoimmune markers or antigens present on specific cells (e.g., cell surface proteins of B cells, T cells, natural killer (NK) cells, bone marrow cells, phagocytes, or tumor cells). Such exogenous receptors can cross-link two kinds of cells and can be used to direct T cells to specific targets and trigger the cytotoxic activity of T cells on the target cells. Examples of such targets can be tumor cells or infectious agents, such as viral pathogens or bacterial pathogens, such as dengue fever virus, herpes simplex virus, influenza virus, HIV or cells carrying autoimmune targets (e.g., IL-2, autoimmune markers or autoimmune antigens).

The main pathways for the intracellular transduction of T cell activation signals include PLC-γ activation pathways and Ras-MAP kinase activation pathways. The cascade reaction of a series of signaling molecules finally leads to the activation of transcription factors (NFAT, NF-kb, AP-1, etc.) and their entrance into the nucleus for regulating the transcription of related target genes.

The “chimeric receptor” as used herein refers to a fusion molecule formed by ligating DNA fragments or protein-corresponding cDNAs from different sources using gene recombination technology, comprising extracellular domain, transmembrane domain and intracellular domain. Chimeric receptors include but are not limited to: chimeric antigen receptor (CAR), modified T cell (antigen) receptor (TCR), T cell fusion protein (TFP), and T cell antigen coupler (TAC).

As used herein, “chimeric antigen receptor” or “CAR” refers to a set of polypeptides that, when in immune effector cells, provide said cells with specificity for target cells (usually cancer cells) and have intracellular signal generation. CAR usually includes at least one antigen binding domain (also referred to as extracellular region), transmembrane domain (also referred to as transmembrane region), and intracellular domain (also referred to herein as “intracellular signaling domain” or “intracellular region”) which comprises functional signaling domains derived from stimulatory molecules and/or costimulatory molecules as defined below. In certain aspects, groups of polypeptides are adjacent to each other. The group of polypeptides comprises a dimerization switch that can couple polypeptides to each other in the presence of dimerization molecules, for example, can couple an antigen binding domain to an intracellular signaling domain. In one aspect, the stimulatory molecule is the zeta chain that binds to the T cell receptor complex. In one aspect, the intracellular domain further comprises one or more functional signaling domains derived from at least one costimulatory molecule as defined below. In one aspect, the costimulatory molecule is selected from the costimulatory molecules described herein, such as 4-1BB (i.e., CD137), CD27, and/or CD28. In one aspect, the CAR comprises a chimeric fusion protein comprising an extracellular antigen binding domain, a transmembrane domain, and an intracellular signaling domain comprising a functional signaling domain derived from a stimulatory molecule. In one aspect, the CAR comprises a chimeric fusion protein comprising an extracellular antigen-binding domain, a transmembrane domain, and an intracellular signaling domain comprising a functional signaling domain derived from a costimulatory molecule and a functional signaling domain derived from a stimulatory molecule. In one aspect, the CAR comprises a chimeric fusion protein comprising an extracellular antigen binding domain, a transmembrane domain, and two functional signaling domains derived from one or more costimulatory molecules.

As used herein, the “transmembrane domain” (also referred to as the transmembrane region) may comprise one or more additional amino acids adjacent to the transmembrane region, such as one or more amino acids associated with the extracellular region of the protein from which the transmembrane protein is derived (for example, 1, 2, 3, 4, 5, 6, 7, 8, 9, 10 up to 15 amino acids in the extracellular region) and/or one or more additional amino acids associated with the extracellular region of the protein from which the transmembrane protein is derived (e.g., 1, 2, 3, 4, 5, 6, 7, 8, 9, 10 up to 15 amino acids in the intracellular region). In one aspect, the transmembrane domain is a domain related to one of the other domains of the chimeric receptor, for example, in one embodiment, the transmembrane domain can be derived from the same protein from which the signaling domain, costimulatory domain, or hinge domain is derived. In certain cases, transmembrane domains can be selected or modified through amino acid substitutions to avoid the binding of such domains to the transmembrane domains of the same or different surface membrane proteins, for example, to minimize the interactions with other members of the receptor complex. In one aspect, the transmembrane domain can homodimerize with another chimeric receptor on the cell surface of the cell expressing the chimeric receptor. In a different aspect, the amino acid sequence of the transmembrane domain can be modified or substituted in order to minimize the interaction with the binding domain of the natural binding partner present in cells expressing the same chimeric receptor. The transmembrane domain can be derived from natural or recombinant sources. When the source is natural, the domain can be derived from any membrane-bound protein or transmembrane protein. In one aspect, the transmembrane domain can transduce signals to the intracellular domain whenever the chimeric receptor binds to the target. The transmembrane domain specifically used in the present invention may include at least the following transmembrane domains: for example, the α, β, or ζ chain of the T-cell receptor, CD28, CD27, CD3ε, CD45, CD4, CD5, CD8, CD9, CD16, CD22, CD33, CD37, CD64, CD80, CD86, CD134, CD137, CD154. In certain embodiments, the transmembrane domain may comprise at least the following transmembrane regions: for example KIRDS2, OX40, CD2, CD27, LFA-1(CD11a, CD18), ICOS(CD278), 4-1BB(CD137), GITR, CD40, BAFFR, HVEM(LIGHTR), SLAMF7, NKp80(KLRF1), NKp44, NKp30, NKp46, CD160, CD19, IL2Rβ, IL2Rγ, IL7Rα, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1(CD226), SLAMF4(CD244, 2B4), CD84, CD96(Tactile), CEACAM1, CRTAM, Ly9(CD229), CD160(BY55), PSGL1, CD100(SEMA4D), SLAMF6(NTB-A, Ly108), SLAM(SLAMF1, CD150, IPO-3), BLAME(SLAMF8), SELPLG(CD162), LTBR, PAG/Cbp, NKG2D, NKG2C.

In some cases, the transmembrane domain may be connected to the extracellular region of the CAR via a hinge (for example, a hinge from a human protein), such as the antigen binding domain of the CAR. For example, in one embodiment, the hinge may be a human Ig (immunoglobulin) hinge (e.g., IgG4 hinge, IgD hinge), GS linker (e.g., GS linker described herein), KIR2DS2 hinge, or CD8a hinge. In one aspect, the transmembrane domain can be recombinant, in which case it will mainly contain hydrophobic residues such as leucine and valine. In one aspect, a triplet of phenylalanine, tryptophan and valine can be found at each end of the recombinant transmembrane domain. Optionally, a short oligopeptide or polypeptide linker between 2 to 10 amino acids in length can form a bond between the transmembrane domain and the cytoplasmic region of the CAR. The glycine-serine dimer provides a particularly suitable linker.

“Intracellular domain” as used herein is generally responsible for the activation of at least one of the normal effector functions of immune cells into which the chimeric receptor has been introduced. The term “effector function” refers to the specialized function of a cell. The effector function of T cells may be, for example, cytolytic activity or auxiliary activity, including secretion of cytokines. Therefore, “intracellular domain” refers to a part of a protein that transduces effector function signals and guides cells to perform specific functions. Although the entire intracellular signaling domain can usually be used, in many cases it is not necessary to use the entire chain. As far as the truncated part of the intracellular signaling domain is used, such a truncated part can be used instead of the complete chain, as long as it transduces effector function signals. Therefore, the term intracellular signaling domain is meant to comprise a truncated part of the intracellular signaling domain sufficient to transduce effector function signals.

It is well known that the signal generated by TCR alone is not sufficient to fully activate T cells, and secondary and/or costimulatory signals are also required. Therefore, T cell activation can be said to be mediated by two different kinds of cytoplasmic signaling sequences: those triggering antigen-dependent primary activation by TCR (primary intracellular signaling domains) and those acting in an antigen-independent manner to provide secondary or costimulatory signals (secondary cytoplasmic domains, such as costimulatory domains).

The term “stimulation” refers to the binding of a stimulatory molecule (for example, TCR/CD3 complex or CAR) to its homologous ligand (or tumor antigen in the case of CAR), thereby mediating a signal transduction event (such as but it is not limited to a signal transduction via the TCR/CD3 complex or a signal transduction via a suitable signaling domain of a NK receptor or a CAR). which induces the primary response. Stimulation can mediate the altered expression of certain molecules.

The term “stimulatory molecule” refers to a molecule expressed by immune cells (for example, T cells, NK cells, B cells) that provides cytoplasmic signaling sequences. The cytoplasmic signaling sequences modulate the activation of immune cells in at least some aspects of the signaling pathways of immune cells in a stimulating manner. In one aspect, the signal is a primary signal initiated by, for example, the binding of a TCR/CD3 complex to a peptide-loaded MEW molecule, and it leads to and mediates T cell responses, including, but not limited to, proliferation, activation, differentiation, and the like. The primary cytoplasmic signaling sequence (also called “primary signaling domain”) that acts in a stimulating manner may comprise the signaling motif called immunoreceptor tyrosine-based activation motif (ITAM). Specifically, examples of ITAM-containing cytoplasmic signaling sequences used in the present invention include, but are not limited to, those derived from the following: CD3ζ, common FcRγ (FCER1G), FcγRIIa, FcRβ (FcEpsilon R1b), CD3γ, CD3δ, CD3ε, CD79a, CD79b, DAP10 and DAP12 which are in the specific CARs of the present invention. The intracellular signaling domains in any one or more CARs of the present invention comprise intracellular signaling sequences, such as primary signaling sequences of CD3-ζ. In the specific CAR of the present invention, the primary signaling sequence of CD3-ζ is the equivalent residue from human or non-human species such as mouse, rodent, monkey, ape, and the like.

The term “costimulatory molecule” refers to a homologous binding partner on T cells, which specifically binds to a costimulatory ligand, thereby mediating the costimulatory response of T cells, such as but not limited to proliferation. Costimulatory molecules are cell surface molecules other than antigen receptors or their ligands, which promote effective immune responses. Costimulatory molecules include but are not limited to MHC class I molecules, BTLA and Toll ligand receptors, as well as OX40, CD27, CD28, CDS, ICAM-1, LFA-1 (CD11a/CD18), ICOS (CD278) and 4-1BB (CD137). Further examples of such costimulatory molecules include CDS, ICAM-1, GITR, BAFFR, HVEM (LIGHTR), SLAMF7, NKp80 (KLRF1), NKp44, NKp30, NKp46, CD160, CD19, CD4, CD8α, CD8β, IL2Rβ, IL2Rγ, IL7Rα, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100(SEMA4D), CD69, SLAMF6(NTB-A, Ly108), SLAM (SLAMF1, CD150, IPO-3), BLAME (SLAMF8), SELPLG (CD162), LTBR, LAT, GADS, SLP-76, PAG/Cbp, CD19a and ligands that specifically bind to CD83.

The costimulatory intracellular signaling domain can be the intracellular part of a costimulatory molecule. The costimulatory molecules can be represented by the following protein families: TNF receptor protein, immunoglobulin-like protein, cytokine receptor, integrin, signaling lymphocyte activation molecule (SLAM protein), and NK cell receptor. Examples of such molecules include CD27, CD28, 4-1BB (CD137), OX40, GITR, CD30, CD40, ICOS, BAFFR, HVEM, ICAM-1, Lymphocyte Function-associated Antigen-1 (LFA-1), CD2, CDS, CD7, CD287, LIGHT, NKG2C, NKG2D, SLAMF7, NKp80, NKp30, NKp44, NKp46, CD160, B7-H3 and ligands that specifically bind to CD83, etc.

The intracellular signaling domain may include all the intracellular part or the entire natural intracellular signaling domain of the molecule, or a functional fragment or derivative thereof.

The term “CD137”, also known as “4-1BB”, refers to a member of the TNFR superfamily with the amino acid sequence provided in GenBank Accession No. AAA62478.2, or from non-human species such as mice, rodents, monkeys, apes, and the like. And “4-1BB costimulatory domain” is defined as the amino acid residues 214˜255 of GenBank Accession No. AAA62478.2, or equivalent residues from non-human species such as mice, rodents, monkeys, apes, and the like. In one aspect, the “4-1BB costimulatory domain” is an equivalent residue from humans or from non-human species such as mice, rodents, monkeys, apes, and the like. For example, the human 4-1BB costimulatory domain has the sequence shown in SEQ ID NO: 34, and the murine 4-1BB costimulatory domain has the sequence shown in SEQ ID NO: 8.

The term “T cell (antigen) receptor (TCR)” is a characteristic mark on the surface of all T cells, which binds to CD3 by non-covalent bonds to form a TCR-CD3 complex. TCR is responsible for recognizing antigens bound to major histocompatibility complex molecules. TCR is a heterodimer composed of two different peptide chains, composed of two peptide chains, a and (3. Each peptide chain can be divided into several parts such as variable region (V region), constant region (C region), transmembrane region and cytoplasmic region; its characteristic is that the cytoplasmic region is very short. TCR molecules belong to the immunoglobulin superfamily, and their antigen specificity exists in the V region; each of the V regions (Vα, Vβ) has three hypervariable regions CDR1, CDR2, and CDR3, among which CDR3 has the largest amount of variation, which directly determines the antigen binding specificity of TCR. When TCR recognizes the MHC-antigen peptide complex, CDR1 and CDR2 recognize and bind to the side wall of the antigen binding groove of the MHC molecule, and CDR3 directly binds to the antigen peptide. TCRs are divided into two categories: TCR1 and TCR2; TCR1 is composed of two chains, γ and δ, and TCR2 is composed of two chains, α and β.

The term “T cell fusion protein (TFP)” comprises recombinant polypeptides derived from various polypeptides that constitute TCR, which can bind to the surface antigens of target cells and interact with other polypeptides of the complete TCR complex. They are usually co-localized on the surface of T cells. TFP is composed of a TCR subunit and an antigen binding domain composed of a human or humanized antibody domain, wherein the TCR subunit comprises at least part of the TCR extracellular domain, transmembrane domain, and stimulatory domain of the intracellular signaling domain of the TCR intracellular domain; the TCR subunit and the antibody domain are effectively connected, wherein the extracellular, transmembrane, and intracellular signaling domains of the TCR subunit are derived from CD3ε or CD3γ, and the TFP is integrated into the TCR expressed on T cells.

The term “T cell antigen coupler (TAC)” includes three functional domains: 1 tumor targeting domain, including single-chain antibody, designed ankyrin repeat protein (DARPin) or other targeting groups; 2 extracellular domain, a single-chain antibody that binds to CD3, leading to the proximity of the TAC receptor and the TCR receptor; 3 the transmembrane region and the intracellular region of the CD4 co-receptor, wherein intracellular region is connected to the protein kinase LCK, which catalyzes the phosphorylation of the immunoreceptor tyrosine activation motifs (ITAMs) of the TCR complex. The phosphorylation acts as the initial step of T cell activation.

The term “antibody” refers to a protein or polypeptide sequence derived from an immunoglobulin molecule that specifically binds to an antigen. Antibodies can be polyclonal or monoclonal, multi-chain or single-chain, or whole immunoglobulins, and can be derived from natural sources or recombinant sources. The antibody may be a tetramer of immunoglobulin molecules.

The term “antibody fragment” refers to at least a portion of an antibody that retains the ability to specifically interact with an epitope of an antigen (e.g., through binding, steric hindrance, stabilization/destabilization, spatial distribution). Examples of antibody fragments include, but are not limited to, Fab, Fab′, F(ab′)₂, Fv fragments, scFv antibody fragments, disulfide-linked Fvs (sdFv), Fd fragments composed of VH and CH1 domains, linear antibodies, single domain antibodies such as sdAb (VL or VH), camelid VHH domains, multispecific antibodies formed by antibody fragments (e.g., bivalent fragments including two Fab fragments connected by disulfide bonds in the hinge region) and isolated CDR or other epitope binding fragments of antibodies. Antigen-binding fragments can also be incorporated into single domain antibodies, maximal antibodies, minibodies, nanobodies, intracellular antibodies, diabodies, tribodies, tetrabodies, v-NAR and double-scFv (see, for example, Hollinger and Hudson, “Nature Biotechnology” (23): 1126-1136, 2005).

The term “scFv” refers to a fusion protein comprising at least one antibody fragment containing a variable region of a light chain and at least one antibody fragment including a variable region of a heavy chain, wherein the variable regions of the light chain and the heavy chain are contiguous (for example, via a synthetic linker such as a short flexible polypeptide linker), and can be expressed as a single-chain polypeptide, and wherein the scFv retains the specificity of the intact antibody from which it is derived. Unless specified, as used herein, the scFv may have the VL and VH variable regions in any order (for example, from the N-terminus and C-terminus of the polypeptide), and the scFv may include VL-linker-VH or may include VH-Linker-VL.

The term “antibody heavy chain” refers to the larger one of the two polypeptide chains present in the antibody molecule in its naturally occurring conformation and usually determining the type that the antibody belongs to.

The term “antibody light chain” refers to the smaller one of the two polypeptide chains present in an antibody molecule in its naturally occurring conformation. κ(k) and λ(l) light chains refer to the two main isotypes of antibody light chains.

The term “recombinant antibody” refers to an antibody produced using recombinant DNA technology, such as, for example, an antibody expressed by a phage or yeast expression system. The term should also be interpreted and referred to antibodies that have been produced by synthesizing a DNA molecule encoding the antibody (and wherein the DNA molecule expresses the antibody protein) or the amino acid sequence of the specified antibody, wherein the DNA or amino acid sequence has been obtained using recombinant DNA technique or amino acid sequence techniques that are available and well-known in the art.

The term “antigen” or “Ag” refers to a molecule that causes an immune response. The immune response may involve the production of antibodies or the activation of cells with specific immunity, or both. Those skilled in the art should understand that any macromolecules including virtually all proteins or peptides can serve as antigens. In addition, antigens can be derived from recombinant or genomic DNAs. When the term is used herein, those skilled in the art should understand that it includes any DNA that comprises a nucleotide sequence or a part of a nucleotide sequence encoding a protein that causes an immune response, and therefore it encodes an “antigen.” In addition, those skilled in the art should understand that the antigen need not to be encoded only by the full-length nucleotide sequence of the gene. It is obvious that the present invention includes, but is not limited to, the use of partial nucleotide sequences of more than one genes, and these nucleotide sequences are arranged in different combinations to encode polypeptides that elicit a desired immune response. Moreover, those skilled in the art should understand that the antigen does not need to be encoded by a “gene” at all. It is obvious that the antigen can be produced synthetically, or it can be derived from a biological sample, or it can be a macromolecule other than a polypeptide. Such biological samples may include, but are not limited to, tissue samples, tumor samples, cells or fluids with other biological components.

“Tumor antigen” refers to a common antigen of a specific hyperproliferative disease. In certain aspects, the antigen of the hyperproliferative disease of the invention is derived from cancer. The tumor antigens of the present invention include but are not limited to: thyroid stimulating hormone receptor (TSHR), CD171, CS-1, C-type lectin-like molecule-1, ganglioside GD3, Tn antigen, CD19, CD20, CD22, CD30, CD70, CD123, CD138, CD33, CD44, CD44v7/8, CD38, CD44v6, B7H3 (CD276), B7H6, KIT (CD117), interleukin 13 receptor subunit α (IL-13Rα), interleukin 11 receptor α (IL-11Rα), prostate stem cell antigen (PSCA), prostate specific membrane antigen (PSMA), carcinoembryonic antigen (CEA), NY-ESO-1, HIV-1 Gag, MART-1, gp100, tyrosinase, mesothelin, EpCAM, protease serine 21 (PRSS21), vascular endothelial growth factor receptor, vascular endothelial growth factor receptor 2 (VEGFR2), Lewis (Y) antigen, CD24, platelet-derived growth factor receptor β (PDGFR-β), stage-specific embryonic antigen-4 (SSEA-4), cell surface-associated mucin 1 (MUC1), MUC6, epidermal growth factor receptor family and the mutants thereof (EGFR, EGFR2, ERBB3, ERBB4, EGFRvIII), nerve cell adhesion molecule (NCAM), carbonic anhydrase IX (CAIX), LMP2, ephrin A receptor 2 (EphA2), fucosyl GM1, sialyl Lewis adhesion molecule (sLe), ganglioside GM3, TGS5, high molecular weight melanoma-associated antigen (HMWMAA), o-acetyl GD2 ganglioside (OAcGD2), folate receptor, tumor vascular endothelial marker 1 (TEM1/CD248), tumor vascular endothelium marker 7 related (TEM7R), Claudin6, Claudin18.2, Claudin18.1, ASGPR1, CDH16, 5T4, 8H9, αvβ6 integrin, B cell maturation antigen (BCMA), CA9, kappa light chain (κ light chain), CSPG4, EGP2, EGP40, FAP, FAR, FBP, embryonic AchR, HLA-A1, HLA-A2, MAGEA1, MAGE3, KDR, MCSP, NKG2D ligand, PSC1, ROR1, Sp17, SURVIVIN, TAG72, TEM1, fibronectin, tenascin, carcinoembryonic variants of tumor necrosis zone, G protein-coupled receptor C group 5-member D (GPRCSD), X chromosome open reading frame 61 (CXORF61), CD97, CD179a, anaplastic lymphoma kinase (ALK), polysialic acid, placenta specific 1 (PLAC1), globoH glycoceramide Hexose part (GloboH), breast differentiation antigen (NY-BR-1), uroplakin 2 (UPK2), hepatitis A virus cell receptor 1 (HAVCR1), adrenaline receptor β3 (ADRB3), pannexin 3 (PANX3), G protein coupled receptor 20 (GPR20), lymphocyte antigen 6 complex locus K9 (LY6K), olfactory receptor 51E2 (OR51E2), TCRγ alternating reading frame protein (TARP), Wilms tumor protein (WT1), ETS translocation variant gene 6 (ETV6-AML), sperm protein 17 (SPA17), X antigen family member 1A (XAGE1), angiogenin-binding cell surface receptor 2 (Tie2), melanoma cancer testis antigen-1 (MAD-CT-1), melanoma cancer testis antigen-2 (MAD-CT-2), Fos-related antigen 1, mutant p53, human telomerase reverse transcriptase (hTERT), sarcoma translocation breakpoint, melanoma inhibitor of apoptosis (ML-IAP), ERG (transmembrane protease serine 2 (TMPRSS2) ETS fusion gene), N-acetylglucosaminyl transferase V (NA17), paired box protein Pax-3 (PAX3), androgen receptor, cyclin B1, V-myc avian myelocytomatosis viral related oncogene, neuroblastoma derived homolog (MYCN), Ras homolog family member C (RhoC), cytochrome P450 1B1 (CYP1B1), CCCTC binding factor (zinc finger protein)-like (BORIS), squamous cell carcinoma antigen recognized by T-cells 3 (SART3), paired box protein Pax-5 (PAX5), proacrosin binding protein sp32 (OYTES1), lymphocyte-specific protein tyrosine kinase (LCK), A kinase anchoring protein 4 (AKAP-4), synovial sarcoma X breakpoint 2 (SSX2), CD79a, CD79b, CD72, leukocyte-related immunoglobulin-like receptor 1 (LAIR1), Fc fragment of IgA receptor (FCAR), leukocyte immunoglobulin-like receptor subfamily member 2 (LILRA2), CD300 molecular-like family member f (CD300LF), C-type lectin domain family 12 member A (CLEC12A), bone marrow stromal cell antigen 2 (BST2), EGF-like module containing, mucin-like, hormone receptor-like 2 (EMR2), lymphocyte antigen 75 (LY75), glypican-3 (GPC3), Fc receptor-like 5 (FCRL5), immunoglobulin λ-like polypeptide 1 (IGLL1).

The pathogen antigen is selected from: the antigen of virus, bacteria, fungus, protozoa, or parasite; the viral antigen is selected from: cytomegaloviral antigen, Epstein-Barr viral antigen, human immunodeficiency viral antigen, or influenza viral antigen.

The term “tumor heterogeneity” means that during the growth of a tumor, after multiple divisions and proliferation, its daughter cells show molecular biological or genetic changes, generating differences in the tumor's growth rate, invasion ability, drug sensitivity, prognosis and other aspects. It is one of the characteristics of malignant tumors.

The term “cancer” refers to a broad category of diseases characterized by hyperproliferative cell growth in vitro (e.g., transformed cells) or in vivo. The conditions that can be treated or prevented by the method of the present invention include, for example, various neoplasms, including benign or malignant tumors, various hyperplasias, and the like. The method of the present invention can achieve the inhibition and/or reversal of the undesirable hyperproliferative cell growth involved in such conditions. Specific examples of cancer include, but are not limited to: breast cancer, blood cancer, colon cancer, rectal cancer, renal cell carcinoma, liver cancer, non-small cell carcinoma of the lung, small intestine cancer, esophagus cancer, melanoma, bone cancer, pancreatic cancer, skin cancer, head and neck cancer, skin or intraocular melanoma, uterine cancer, ovarian cancer, rectal cancer, anal cancer, stomach cancer, testicular cancer, uterine cancer, fallopian tube cancer, endometrial cancer, cervical cancer, vaginal cancer, vulvar cancer, Hodgkin's disease, non-Hodgkin's lymphoma, endocrine system cancer, thyroid cancer, parathyroid cancer, adrenal cancer, soft tissue sarcoma, urethral cancer, penis cancer, pediatric solid tumor, bladder cancer, renal or ureteral cancer, renal pelvic cancer, central nervous system (CNS) tumor, primary CNS lymphoma, tumor angiogenesis, spinal tumor, glioma, pituitary adenoma, Kaposi's sarcoma, epidermoid carcinoma, squamous cell carcinoma, T-cell lymphoma, environmentally induced cancers, combinations and metastasis thereof.

The term “transfected” or “transformed” or “transduced” refers to the process by which exogenous nucleic acid is transferred or introduced into a host cell. A “transfected” or “transformed” or “transduced” cell is a cell that has been transfected, transformed or transduced with an exogenous nucleic acid. The cells include subjects' primary cells and their progeny.

The term “specifically binding” refers to that antibodies or ligands recognize and bind to a protein of a binding partner (such as a tumor antigen) present in a sample, but the antibodies or ligands basically do not recognize or bind to other molecules in the sample.

“Refractory” as used herein refers to that a disease, such as cancer, does not respond to treatments. In an embodiment, the refractory cancer may be resistant to treatments before or at the beginning of treatments. In other embodiments, refractory cancer may become resistant during the treatments. Refractory cancer is also called resistant cancer. In the present invention, refractory cancers include, but are not limited to, cancers that are not sensitive to radiotherapy, that relapse after radiotherapy, that are not sensitive to chemotherapy, that relapse after chemotherapy, that are not sensitive to CAR-T treatment, or that relapse after CAR-T treatment. The treatment regimens described herein can be used to refractory or recurrent malignant tumors.

“Relapsed” as used herein refers to the return of a disease such as a cancer or the signs and symptoms of a disease (e.g., cancer) after a period of improvement, for example, a therapy, such as a previous treatment of a cancer therapy.

The terms “individual” and “subject” have the same meaning herein, and can be humans and animals from other species.

The term “enhancement” refers to allowing a subject or tumor cell to improve its ability to respond to the treatment disclosed herein. For example, an enhanced response may include 5%, 10%, 15%, 20%, 25%, 30%, 35%, 40%, 45%, 50%, 55%, 60%, 65%, 70%, 75%, 80%, 85%, 90%, 95% or 98% or more increase in responsiveness. As used herein, “enhancing” can also refer to increasing the number of subjects responding to the treatment, such as immune effector cell therapy. For example, an enhanced response can refer to the total percentage of subjects responding to the treatment, wherein the percentages are 5%, 10%, 15%, 20%, 25%, 30%, 35%, 40%, 45%, 50%, 55%, 60%, 65%, 70%, 75%, 80%, 85%, 90%, 95% or 98%, or more.

In one aspect, the treatment is determined by clinical outcomes: the increase, enhancement or extension of the anti-tumor activity of T cells; the increase in the number of anti-tumor T cells or activated T cells compared with the number of which before treatment; the promotion of IFN-γ secretion; or a combination thereof. In another aspect, the clinical outcome is tumor regression; tumor shrinkage; tumor necrosis; anti-tumor response by the immune system; tumor enlargement, recurrence or spread, or a combination thereof. In an additional aspect, the therapeutic effect is predicted by the presence of T cells, the presence of genetic markers indicating T cell inflammation, the promotion of IFN-γ secretion, or a combination thereof.

The immune effector cells as disclosed herein can be administered to individuals by various routes, including, for example, orally or parenterally, such as intravenous, intramuscular, subcutaneous, intraorbital, intrasaccular, intraperitoneal, intrarectal, intracisternal, intratumoral, intranasally, intradermally, or passive or promoted absorption through the skin using, for example, skin patches or transdermal iontophoresis, respectively.

The total amount of agent to be administered in practicing the method of the present invention can be administered to a subject as a single dose by a bolus injection or by an infusion in a relatively short period of time, or can be administered using a graded treatment regimen, wherein multiple doses are administered over an extended period of time. Those skilled in the art will know that the amount of the composition for treating a pathological condition in a subject depends on many factors, including the age and general health of the subject, as well as the route of administration and the number of treatments to be administered. Taking these factors into account, the technician will adjust the specific dosage as needed. Generally, initially, phase I and phase II clinical trials are used to determine the formulation and the administration route and frequency of the composition.

Range: Throughout this disclosure, each aspects of the invention can be presented in a range format. It should be understood that the description in range format is merely for convenience and brevity, and should not be regarded as an unchangeable limitation on the scope of the present invention. Therefore, the description of a range should be considered as that all possible sub-ranges and individual values within that range are specifically disclosed. For example, a description of a range such as from 1 to 6 should be considered to specifically disclose subranges such as 1 to 3, 1 to 4, 1 to 5, 2 to 4, 2 to 6, 3 to 6, etc., and individual values within the range, such as 1, 2, 2.7, 3, 4, 5, 5.3, and 6. As another example, a range such as 95-99% identity includes a range with 95%, 96%, 97%, 98%, or 99% identity, and includes sub-ranges such as 96-99%, 96-98%, 96-97%, 97-99%, 97-98% and 98-99% identity. This applies regardless of the width of the range.

Based on the present disclosure, those skilled in the art should understand that many changes or modifications can be made to the disclosed specific embodiments and still obtain the same or similar results without departing from the spirit and scope of the present invention. The scope of the present invention is not limited to the specific embodiments described herein (which are only intended to illustrate various aspects of the invention), and functionally equivalent methods and components are within the scope of the present invention. In fact, the various modifications of the present invention plus those shown and described herein will become apparent to those skilled in the art based on the foregoing description.

The term “CLD18” refers to claudin-18, including any variants (including CLD18A1 (claudin 18.1) and CLD18A2 (claudin 18.2)), conformations, isoforms, and species homologs of CLD18 expressed by cells naturally expressed or transfected with the CLD18 gene. Preferably, “CLD18” refers to human CLD18, particularly CLD18A2 (SEQ ID NO: 28) and/or CLD18A1 (SEQ ID NO: 27), more preferably CLD18A2.

In a specific embodiment, the chimeric antigen receptor polypeptides of the present invention can be selected from those sequentially connected in the following manners:

extracellular antigen binding region-CD8 transmembrane region-4-1BB-CD3ζ,

extracellular antigen binding region-CD8 transmembrane region-CD28b-CD3ζ,

extracellular antigen binding region-CD28a-CD28b-CD3ζ,

extracellular antigen binding region-CD28a-CD28b-4-1BB-CD3ζ,

and a combination thereof, wherein the CD28a in the relevant chimeric antigen receptor protein represents the transmembrane region of the CD28 molecule, and CD28b represents the intracellular signaling region of the CD28 molecule.

The present invention also comprises a nucleic acid encoding the chimeric antigen receptor. The present invention also relates to variants of the above-mentioned polynucleotides, which encode polypeptides having the same amino acid sequence as the present invention, or fragments, analogs and derivatives thereof.

The present invention also provides a vector comprising the nucleic acid of the above-mentioned chimeric antigen receptor. The present invention also comprises viruses comprising the above-mentioned vectors. The viruses of the present invention comprise packaged infectious viruses, and also comprises viruses to be packaged that comprise the necessary components for packaging infectious viruses. Other viruses known in the art for transducing foreign genes into immune effector cells and their corresponding plasmid vectors can also be used in the present invention.

The present invention also provides a chimeric antigen-modified immune effector cell, which is transduced with a nucleic acid encoding the chimeric antigen receptor or is transduced with the aforementioned recombinant plasmid containing the nucleic acid, or a virus containing the plasmid. Conventional nucleic acid transduction methods in the art, including non-viral and viral transduction methods, can be used in the present invention. Non-viral-based transduction methods include electroporation and transposon methods. The Nucleofector nuclear transfection instrument developed by Amaxa recently can directly introduce foreign genes into the nucleus to obtain efficient transduction of the target gene. In addition, the transduction efficiencies of transposon systems based on Sleeping Beauty system or PiggyBac transposon are much higher than that of ordinary electroporations. The combined application of nucleofector transfection instrument and Sleeping Beauty transposon system has been reported [Davies JK., et al. Combining CD19 redirection and alloanergization to generate tumor-specific human T cells for allogeneic cell therapy of B-cell malignancies. Cancer Res, 2010, 70(10): OF1-101, this method not only has high transduction efficiency but also can realize the targeted integration of the target gene. In one embodiment of the present invention, the method for transducing immune effector cells to achieve the modification by chimeric antigen receptor gene is based on the method for transducing viruses such as retroviruses or lentiviruses. The method has the advantages of high transduction efficiency and stable expression of foreign genes, and shortens the in vitro culture time for the number of immune effector cells to reach the clinical standards. On the surface of the transgenic immune effector cell, the transduced nucleic acid is expressed on its surface through transcription and translation. Through in vitro cytotoxicity experiments on various cultured tumor cells, it is proved that the immune effector cells modified by the chimeric antigen of the present invention have highly specific killing effects on tumor cells (also known as cytotoxicity), and can effectively survive in tumor tissues. Therefore, the nucleic acid encoding the chimeric antigen receptor, the plasmid containing the nucleic acid, the virus containing the plasmid, and the transgenic immune effector cells transduced with the nucleic acid, plasmid or virus of the present invention can be effectively used for tumor immunotherapy.

The chimeric antigen-modified immune effector cells of the present invention can also express another chimeric receptor besides the above-mentioned chimeric receptor, which does not contain CD3ζ, but contains the intracellular signaling domain of CD28, the intracellular signaling domain of CD137 or a combination of the two.

The immune effector cells modified by the chimeric antigen receptor of the present invention can be applied to the preparation of pharmaceutical compositions or diagnostic reagents. In addition to an effective amount of the immune cells, the composition may also comprise a pharmaceutically acceptable carrier. The term “pharmaceutically acceptable” means that when the molecular entities and the compositions are properly administered to animals or humans, they will not produce adverse, allergic or other adverse reactions.

Specific examples of some substances that can be used as pharmaceutically acceptable carriers or components thereof are sugars, such as lactose, glucose, and sucrose; starches, such as corn starch and potato starch; cellulose and its derivatives, such as carboxymethyl fiber sodium, ethyl cellulose and methyl cellulose; tragacanth powder; malt; gelatin; talc; solid lubricants, such as stearic acid and magnesium stearate; calcium sulfate; vegetable oils, such as peanut oil, cottonseed oil, sesame oil, olive oil, corn oil, and cocoa butter; polyols, such as propylene glycol, glycerin, sorbitol, mannitol, and polyethylene glycol; alginic acid; emulsifiers, such as Tween®; wetting agents, such as sodium lauryl sulfate; coloring agent; flavoring agent; tabletting agent, stabilizer; antioxidant; preservative; pyrogen-free water; isotonic salt solution and phosphate buffer, etc.

The composition of the present invention can be made into various dosage forms according to needs, and a physician can determine the beneficial dosage for a patient according to factors such as the patient's type, age, weight, general disease condition, and administration method. The method of administration can be injection or other treatment methods.

Advantages of the Present Invention:

1. The immune effector cells modified by the chimeric antigen receptor of the present invention can effectively increase the proliferation, survival and function of the immune effector cells in tumors; after overexpressing cytokines IL21 and CCL19, the expression of these inhibitory immune checkpoints (PD-1, LAG-3, TIM-3) of the immune effector cells modified by the chimeric antigen receptor can be reduced, thereby alleviating the depletion of T cells.

2. The immune effector cells modified by the chimeric antigen receptor of the present invention not only are effective against solid tumor cells in vitro, but compared with the immune effector cells modified by chimeric antigen receptors that do not co-express IL-21 and CCL19, have better in vivo killing effect on solid tumor cells and in vitro expansion performance.

The present invention will be further explained below with reference to specific examples. It should be understood that these examples are only used to illustrate the present invention and not to limit the scope of the present invention. The experimental methods without specific conditions in the following examples usually follow the conventional conditions as described in J. Sambrook et al., Molecular Cloning Experiment Guide, the Third Edition, Science Press, 2002, or follow the conditions recommended by the manufacturer.

Exemplary antigen receptors of the present invention, including CAR, and methods for engineering and introducing receptors into cells, refer to, for example, those disclosed in Chinese Patent Application Publication Nos. CN107058354A, CN107460201A, CN105194661A, CN105315375A, CN105713881A, CN106146666A, CN106519037A, CN106554414A, CN105331585A, CN106397593A, CN106467573A, CN104140974A, International Patent Application Publication Nos.: WO2017186121A1, WO2018006882A1, WO2015172339A8, WO2018/018958A1. In the following examples, UTD usually means blank T cells, that is, uninfected T cells.

### Example 1. Construction of T Cells Expressing Chimeric Antigen Receptors

1. Plasmid Construction

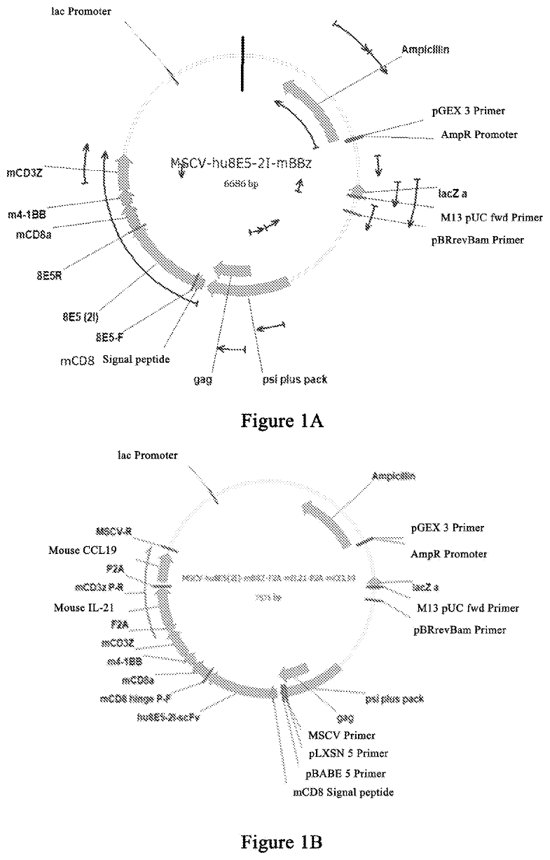

Conventional molecular biology methods in the art are used. The scFv used in this example is an antibody targeting claudin18.2. The nucleic acid sequence is shown in SEQ ID NO: 1, and the amino acid sequence is shown in SEQ ID NO: 2. Exemplarily, the chimeric antigen receptor used in the examples is a second-generation chimeric antigen receptor, which has a transmembrane domain of CD8, an intracellular domain of 4-1BB, and a CD3ζ. Referring to the plasmid map shown in FIG. 1, the plasmid MSCV-hu8E5-2I-mBBZ is constructed.

Using MSCV.pBABE 5 (purchased from addgene) as a vector, a retroviral plasmid MSCV-hu8E5-2I-mBBZ expressing the second-generation chimeric antigen receptor is constructed (the plasmid construction map is shown in FIG. 1A). The sequence of hu8E5-2I-mBBZ consists of mouse CD8α signal peptide (SEQ ID NO: 3), scFv targeting claudin 18.2 (SEQ ID NO: 1), mouse CD8 hinge and transmembrane region (SEQ ID NO: 5) and mouse 4-1BB intracellular signaling domain (SEQ ID NO: 7) and intracellular segment CD3ζ of mouse CD3 (SEQ ID NO: 9).

The F2A-mIL-21-P2A-mCCL19 sequence is inserted into the MSCV-hu8E5-2I-mBBZ plasmid, and the chimeric antigen receptor targeting Claudin 18.2 and the corresponding retroviral plasmid MSCV-hu8E5-2I-mBBZ-F2A-mIL-21-P2A-mCCL19 are constructed (the plasmid construction map is shown in FIG. 1B). F2A-mIL-21-P2A-mCCL19 consists of F2A (SEQ ID NO: 11), mouse IL21 (SEQ ID NO: 13), P2A (SEQ ID NO: 15), and mouse CCL19 (SEQ ID NO: 17).

2. Virus Transfection

MSCV-hu8E5-2I-mBBZ, MSCV-hu8E5-2I-mBBZ-F2A-mIL-21-P2A-mCCL19 are respectively transfected into 293T cells to obtain retroviruses MSCV-hu8E5-2I-mBBZ and MSCV-hu8E5-2I-mBBZ-F2A-mIL-21-P2A-mCCL19.

3. Preparation of CAR-T Cells

Take T lymphocytes from the spleen of C57BL/6 mice, add the purified mouse CD3+T lymphocytes to Dynabeads Mouse T-activator CD3/CD28 at a volume ratio of 1:1, wash them once with PBS, activate them, and culture them in an incubator. The culture medium is RPMI 1640 complete medium, supplemented with 10% FBS serum. The T lymphocytes from the spleen of mice are activated for 24 h and inoculated into a 12-well plate coated with recombinant human fibrin fragments, and MSCV-hu8E5-2I-mBBZ, MSCV-hu8E5-2I-mBBZ-F2A-mIL-21-P2A-mCCL19 retrovirus are added respectively to infect the lymphocytes for 12 hours. It is followed by culture and expansion of the lymphocytes to the required number to obtain mouse hu8E5-2I-mBBZ CAR T cells (mBBZ CAR T), and hu8E5-2I-mBBZ-F2A-mIL-21-P2A-mCCL19 CAR T cells (mBBZ-21*19 CAR T).

### Example 2 In Vitro Cytokine Detection

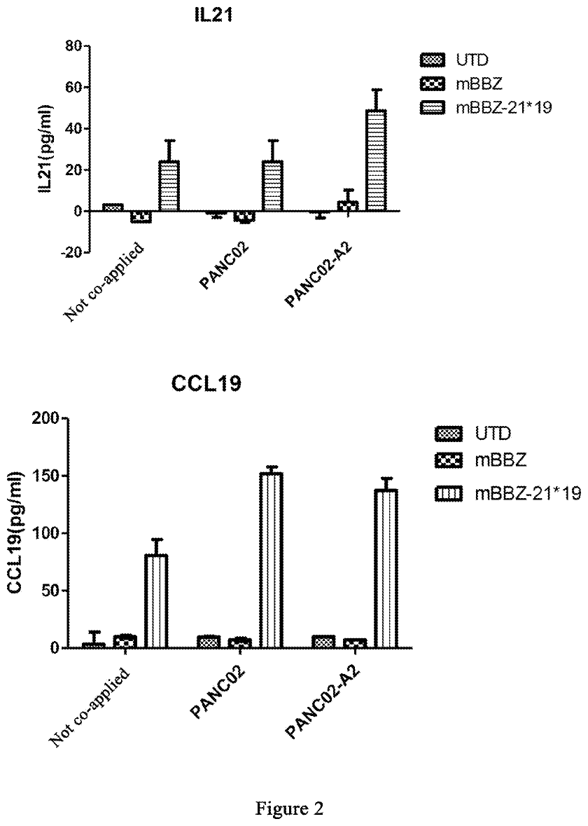

First, use mitomycin C to pretreat mouse pancreatic cancer target cells PANC02 (negative expression of claudin 18.2, purchased from ATCC) and PANC02-A2 (positive expression of claudin 18.2), inoculate 2×10⁵ cells/400 μl in a 24-well plate, and inoculate Untransduced (UTD) T cells, mBBZ CAR T cells, mBBZ-21*19 CAR T cells in a 24-well plate at the effector to target cell ratio of 1:1. Wherein a control group without target cells is set up. The cell supernatants are collected after the third day, and the secretions of each cytokines, mIL21 and mCCL19, are detected by an ELISA kit. The results are shown in FIG. 2. It can be seen from FIG. 2 that mBBZ-21*19 CAR-T can secrete cytokines IL21 and CCL19, and does not depend on antigen stimulation.

### Example 3 In Vitro CAR-T Cell Phenotype Detection

Take UTD, mBBZ CAR T cells and mBBZ-21*19 CAR T cells infected for four days for cell surface immune checkpoint (PD-1, LAG-3, TIM-3) detection. First, collect different CAR-T cells in EP tubes. Cells of each type are divided into 3 tubes, and washed twice with a pre-ice-bathed flow washing solution (1% NCS plus PBS), and different detection tubes are added with BV421-labeled anti-PD-1 antibodies, APC-labeled anti-LAG-3 antibodies, and APC-labeled anti-TIM-3 antibodies at a 1:50-proportion dilution, incubated on ice for 45 minutes, washed 3 times for flow cytometry.

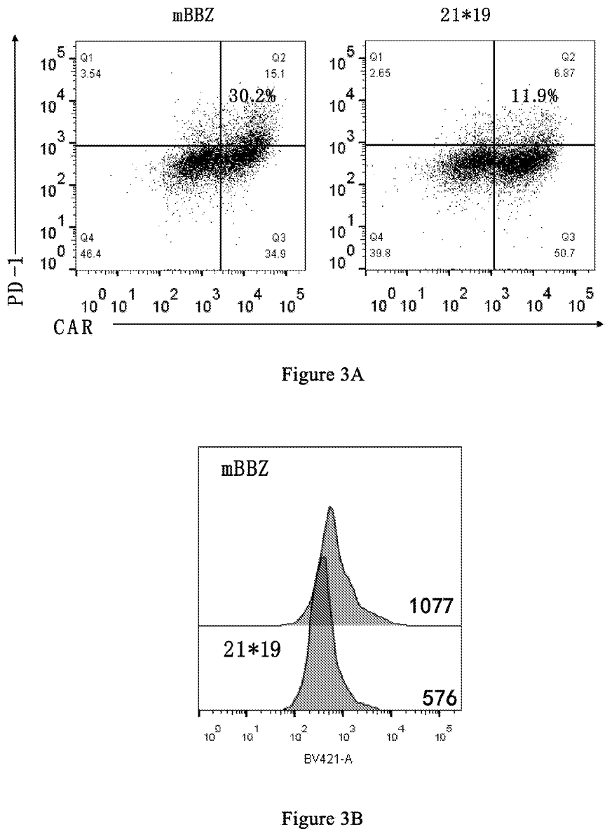

FIG. 3A shows the secretion of PD-1 by cells in different groups. The results show that the secretion of PD-1 in the mBBZ group reaches 30.2%, and the secretion of PD-1 in the mBBZ-21*19 group is only 11.9%. From FIG. 3B, the expression of PD-1 in the mBBZ group is higher than that of mBBZ-21*19.

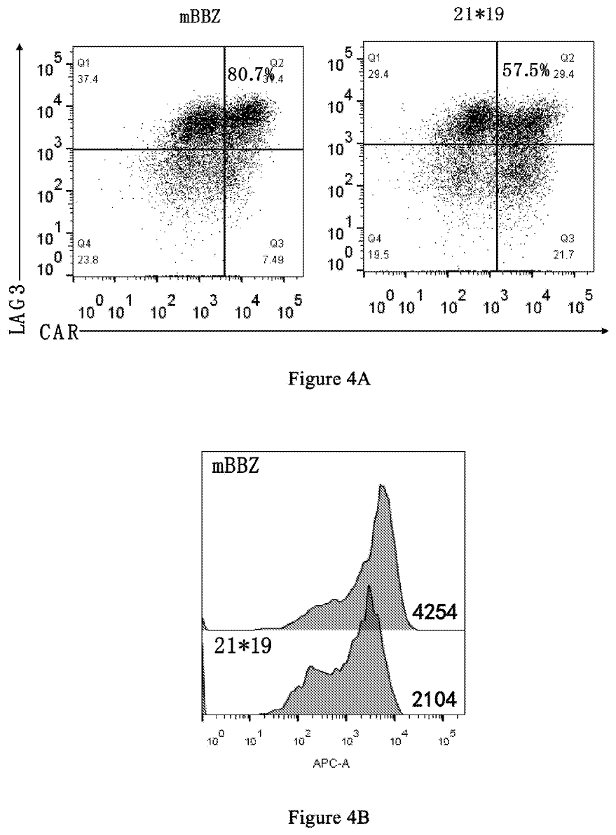

FIG. 4A shows the secretion of LAG-3 by cells in different groups. The results show that the secretion of LAG-3 in the mBBZ group reaches 80.7%, and the secretion of LAG-3 in the mBBZ-21*19 group is 57.5%. From FIG. 4B, the expression of LAG-3 in the mBBZ group is higher than that in the mBBZ-21*19 group.

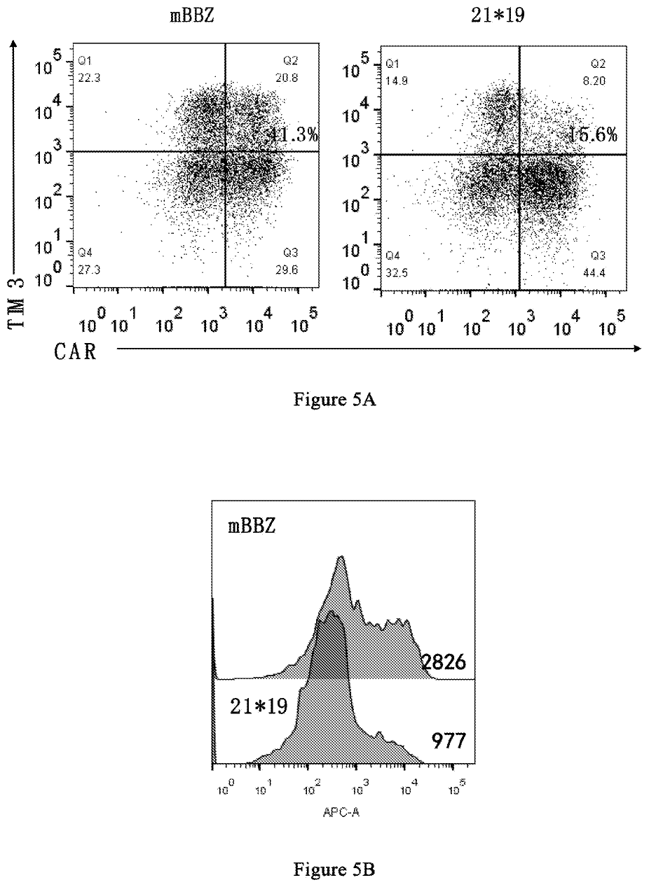

FIG. 5A shows the secretion of TIM-3 by cells in different groups. The results show that the secretion of TIM-3 in the mBBZ group reaches 41.3%, and the secretion of TIM-3 in the mBBZ-21*19 group is only 15.6%. From FIG. 5B, the expression of TIM-3 in the mBBZ group is higher than that in the mBBZ-21*19 group.

In summary, the expressions of PD-1, LAG-3 and TIM-3 on the surface of mBBZ-21*19 CAR T cells are lower than that of mBBZ CAR T cells. Since inhibitory immune checkpoints play an important role in tumor immunosuppression, the results can show that overexpression of cytokines IL21 and CCL19 can reduce the expression of these inhibitory immune checkpoints, thereby alleviating the depletion of T cells.

### Example 4. In Vitro Killing Toxicity Test

CytoTox 96 non-radioactive cytotoxicity detection kit (Promega) is used. The specific method follows the instructions of CytoTox 96 non-radioactive cytotoxicity detection kit.

Effector cells: inoculate UTD cells, mBBZ CAR T cells, mBBZ-21*19 CAR T cells in 96-well plates at the effector to target cells ratios of 3:1, 1:1 or 1:3, respectively.

Target cells: inoculate 50 μL of 2×10⁵/mL mouse pancreatic cancer cell lines PANC02-A2 and PANC02 cells to the corresponding 96-well plates, respectively.

Each group has 5 holes as duplications. Place the culture plate in the cell culture box and incubate for 18 hours.

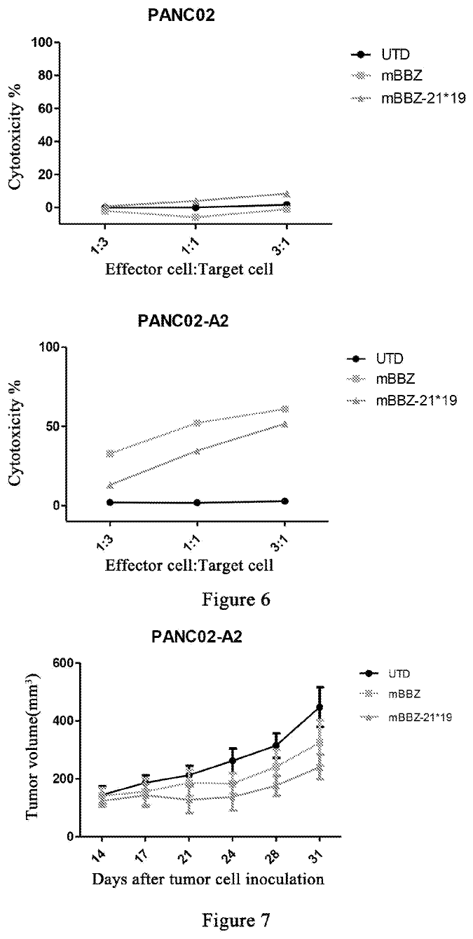

The experimental groups and the control groups are set as follows: experimental group: each target cell+T lymphocyte expressing different chimeric antigen receptors; control group 1: target cell with maximum LDH release; control group 2: target cells that spontaneously release LDH; control group 3: effector cells that spontaneously release LDH. The calculation formula is: % cytotoxicity=[(experimental group−spontaneous effector cell group−spontaneous target cell group)/(target cell with maximum LDH−spontaneous target cell)]*100. The experimental results are shown in FIG. 6.

As shown in FIG. 6, mBBZ CAR T cells and mBBZ-21*19 CAR T, compared with control group UTD, have significant toxic killing effects on PANC02-A2 positively expressing claudin 18.2 at the effector to target cells ratios of 3:1 and 1:1; and have almost no killing effects on PANC02 cells negatively expressing claudin 18.2.

### Example 5. Anti-Tumor Treatment Experiment on Subcutaneous Xenograft Tumor

PANC02-A2 Subcutaneous Transplanted Tumor Model

1) Experimental grouping: C57BL/6 mice of 6-8 weeks' old are randomly divided into groups, 5-6 mice in each group, the groups are UTD cell, mBBZ CAR T cell, and mBBZ-21*19 CAR T cell treatment groups.

2) Inoculation of subcutaneous transplanted tumor: using trypsin digestion method to collect PANC02-A2 cells in logarithmic growth phase and in good growth state. After washing once with PBS, the cell density is adjusted to 6×10⁶/mL, and a syringe is used to inject 200 μL of cell suspension into the subcutaneous part of the right abdomen of C57BL/6 mice, that is, each mouse is inoculated with 1.2×10⁶ tumor cells, and the inoculation diary records it as day 0.

3) CAR-T cell reinfusion: D11 day after subcutaneous inoculation of the tumor cells, the average tumor volume is about 150 mm³. Inject CART cells, at the injection dose: 5×10⁶/mouse.

The results are shown in FIG. 7. 17 days after CAR-T injection, compared with the UTD control group, each group has a significant tumor suppressing effect. The inhibition rates are: mBBZ CAR T group: 38%, mBBZ-21*19 CAR T group: 46.2%. It shows that the anti-tumor effect of mBBZ-21*19 CART cell treatment group is better than that of mBBZ CART cell.

Exemplarily, the above examples select CAR-T cells that target claudin 18.2. It should be understood that CAR-T cells that act on other targets, such as GPC3, EGFR, EGFRvIII, CD19, BCMA, also have the same effects. The antibodies used can be murine antibodies or humanized antibodies.

Exemplarily, the above examples select a mouse-derived CAR, but its signal peptide, hinge region, transmembrane region, etc. can be selected from other species, including but not limited to human signal peptide, hinge region, transmembrane domain, and intracellular region, according to different purposes. Antibodies can also be selected according to different purposes. Mouse antibodies or humanized antibodies or complete human antibodies against different targets can be selected. For example, the transmembrane region of human CD28 (the amino acid sequence is shown in SEQ ID NO: 31), the intracellular region of human CD28 (the amino acid sequence is shown in SEQ ID NO: 32), the intracellular region of human 41BB (the amino acid sequence is shown in SEQ ID NO: 34), the intracellular region of human CD3 (the amino acid sequence is shown in SEQ ID NO: 33), and the transmembrane domain of human CD8.

Exemplarily, although CAR-T cells are used in the above example, other immune cells, such as NK cells, NK-T cells, and specific subtypes of immune cells, such as γ/δ T cells, can also be specifically selected.

Exemplarily, the above examples select a mouse-derived CAR, but its signal peptide, hinge region, transmembrane region, etc. can be selected from other species according to different purposes. It includes but is not limited to human signal peptide, hinge region, transmembrane domain, and intracellular region. Antibodies can also be selected according to different purposes. Mouse antibodies or humanized antibodies or complete human antibodies against different targets can be selected.

The sequences involved in the present invention are shown in the following table:

| SEQ | | |
|---|---|---|
| ID | | |
| NO. | NAME | SEQUENCE |
| 1 | Nucleic acid | CAGGTGCAGCTGCAGGAGAGCGGCCCCGGCCTGATCAAGCCCAGCCAGACCCTGAGCCTGAC |
| | sequence of | CTGCACCGTGAGCGGCGGCAGCATCAGCAGCGGCTACAACTGGCACTGGATCCGGCAGCCCC |
| | Hu8E5-2I | CCGGCAAGGGCCTGGAGTGGATCGGCTACATCCACTACACCGGCAGCACCAACTACAACCCC |
| | scFV | GCCCTGCGGAGCCGGGTGACCATCAGCGTGGACACCAGCAAGAACCAGTTCAGCCTGAAGCT |
| | | GAGCAGCGTGACCGCCGCCGACACCGCCATCTACTACTGCGCCCGGATCTACAACGGCAACAG |
| | | CTTCCCCTACTGGGGCCAGGGCACCACCGTGACCGTGAGCAGCGGTGGAGGCGGTTCAGGCG |
| | | GAGGTGGTTCTGGCGGTGGCGGATCGGACATCGTGATGACCCAGAGCCCCGACAGCCTGGCC |
| | | GTGAGCCTGGGCGAGCGGGCCACCATCAACTGCAAGAGCAGCCAGAGCCTGTTCAACAGCGG |
| | | CAACCAGAAGAACTACCTGACCTGGTACCAGCAGAAGCCCGGCCAGCCCCCCAAGCTGCTGA |
| | | TCTACTGGGCCAGCACCCGGGAGAGCGGCGTGCCCGACCGGTTCAGCGGCAGCGGCAGCGGC |
| | | ACCGACTTCACCCTGACCATCAGCAGCCTGCAGGCCGAGGACGTGGCCGTGTACTACTGCCAG |
| | | AACGCCTACAGCTTCCCCTACACCTTCGGCGGCGGCACCAAGCTGGAGATCAAGCGG |
| 2 | Amino acid | QVQLQESGPGLIKPSQTLSLTCTVSGGSISSGYNWHWIRQPPGKGLEWIGYIHYTGSTNYNPALRSR |
| | sequence of | VTISVDTSKNQFSLKLSSVTAADTAIYYCARIYNGNSFPYWGQGTTVTVSSGGGGSGGGGSGGGG |
| | Hu8E5-2I | SDIVMTQSPDSLAVSLGERATINCKSSQSLFNSGNQKNYLTWYQQKPGQPPKLLIYWASTRESGVP |
| | scFV | DRFSGSGSGTDFTLTISSLQAEDVAVYYCQNAYSFPYTFGGGTKLEIKR |
| 3 | mouse CD8α | ATGGCCTCACCGTTGACCCGCTTTCTGTCGCTGAACCTGCTGCTGCTGGGTGAGTCGATTATCC |
| | signal peptide | TGGGGAGTGGAGAAGCT |
| 4 | Amino acid | MASPLTRFLSLNLLLLGESIILGSGEA |
| | sequence of | |
| | mouse CD8α | |
| | signal peptide | |
| 5 | mouse CD8 | ACTACTACCAAGCCAGTGCTGCGAACTCCCTCACCTGTGCACCCTACCGGGACATCTCAGCCC |
| | hinge + | CAGAGACCAGAAGATTGTCGGCCCCGTGGCTCAGTGAAGGGGACCGGATTGGACTTCGCCTG |
| | transmembrane | TGATATTTACATCTGGGCACCCTTGGCCGGAATCTGCGTGGCCCTTCTGCTGTCCTTGATCATCA |
| | domain | CTCTCATCTGCTACCACAGGAGCCGA |
| 6 | Amino acid | TTTKPVLRTPSPVHPTGTSQPQRPEDCRPRGSVKGTGLDFACDIYIWAPLAGICVALLLSLIITLICYH |
| | sequence of | RSR |
| | mouse CD8 | |
| | hinge + | |
| | transmembrane | |
| | domain | |
| 7 | Nucleotide | AAATGGATCAGGAAAAAATTCCCCCACATATTCAAGCAACCATTTAAGAAGACCACTGGAGCA |
| | sequence of | GCTCAAGAGGAAGATGCTTGTAGCTGCCGATGTCCACAGGAAGAAGAAGGAGGAGGAGGAG |
| | mouse 4-1BB | GCTATGAGCTG |
| | intracellular | |
| | domain | |
| 8 | Amino acid | KWIRKKFPHIFKQPFKKTTGAAQEEDACSCRCPQEEEGGGGGYEL |
| | sequence of | |
| | mouse 4-1BB | |
| | intracellular | |
| | domain | |
| 9 | Intracellular | AGCAGGAGTGCAGAGACTGCTGCCAACCTGCAGGACCCCAACCAGCTCTACAATGAGCTCAA |
| | segment | TCTAGGGCGAAGAGAGGAATATGACGTCTTGGAGAAGAAGCGGGCTCGGGATCCAGAGATGG |
| | CD3ξ of | GAGGCAAACAGCAGAGGAGGAGGAACCCCCAGGAAGGCGTATACAATGCACTGCAGAAAGA |
| | mouse CD3 | CAAGATGGCAGAAGCCTACAGTGAGATCGGCACAAAAGGCGAGAGGCGGAGAGGCAAGGGG |
| | | CACGATGGCCTTTACCAGGGTCTCAGCACTGCCACCAAGGACACCTATGATGCCCTGCATATGC |
| | | AGACCCTGGCC |
| 10 | Amino acid | SRSAETAANLQDPNQLYNELNLGRREEYDVLEKKRARDPEMGGKQQRRRNPQEGVYNALQKDK |
| | sequence of | MAEAYSEIGTKGERRRGKGHDGLYQGLSTATKDTYDALHMQTLA |
| | the | |
| | intracellular | |
| | segment | |
| | CD3ξ of | |
| | mouse CD3 | |
| 11 | Nucleotide | GTGAAACAGACTTTGAATTTTGACCTTCTGAAGTTGGCAGGAGACGTTGAGTCCAACCCTGGG |
| | sequence of | CCC |
| | F2A | |
| 12 | Amino acid | VKQTLNFDLLKLAGDVESNPGP |
| | sequence of | |
| | F2A | |
| 13 | Nucleotide | ATGGAGAGGACCCTTGTCTGTCTGGTAGTCATCTTCTTGGGGACAGTGGCCCATAAATCAAGCC |
| | sequence of | CCCAAGGGCCAGATCGCCTCCTGATTAGACTTCGTCACCTTATTGACATTGTTGAACAGCTGAA |
| | mouse IL21 | AATCTATGAAAATGACTTGGATCCTGAACTTCTATCAGCTCCACAAGATGTAAAGGGGCACTGT |
| | | GAGCATGCAGCTTTTGCCTGTTTTCAGAAGGCCAAACTCAAGCCATCAAACCCTGGAAACAAT |
| | | AAGACATTCATCATTGACCTCGTGGCCCAGCTCAGGAGGAGGCTGCCTGCCAGGAGGGGAGG |
| | | AAAGAAACAGAAGCACATAGCTAAATGCCCTTCCTGTGATTCGTATGAGAAAAGGACACCCAA |
| | | AGAATTCCTAGAAAGACTAAAATGGCTCCTTCAAAAGATGATTCATCAGCATCTCTCC |
| 14 | Amino acid | MERTLVCLVVIFLGTVAHKSSPQGPDRLLIRLRHLIDIVEQLKIYENDLDPELLSAPQDVKGHCEHA |
| | sequence of | AFACFQKAKLKPSNPGNNKTFIIDLVAQLRRRLPARRGGKKQKHIAKCPSCDSYEKRTPKEFLERL |
| | mouse IL21 | KWLLQKMIHQHLS |
| 15 | Nucleotide | GCTACTAACTTCAGCCTGCTGAAGCAGGCTGGAGACGTGGAGGAGAACCCTGGACCT |
| | sequence of | |
| | P2A | |
| 16 | Amino acid | ATNFSLLKQAGDVEENPGP |
| | sequence of | |
| | P2A | |
| 17 | Nucleotide | ATGGCCCCCCGTGTGACCCCACTCCTGGCCTTCAGCCTGCTGGTTCTCTGGACCTTCCCAGCCC |
| | sequence of | CAACTCTGGGGGGTGCTAATGATGCGGAAGACTGCTGCCTGTCTGTGACCCAGCGCCCCATCC |
| | mouse CCL19 | CTGGGAACATCGTFAAAGCCTTCCGCTACCTTCTTAATGAAGATGGCTGCAGGGTGCCTGCTGT |
| | | TGTGTTCACCACACTAAGGGGCTATCAGCTCTGTGCACCTCCAGACCAGCCCTGGGTGGATCG |
| | | CATCATCCGAAGACTGAAGAAGTCTTCTGCCAAGAACAAAGGCAACAGCACCAGAAGGAGCC |
| | | CTGTGTCT |
| 18 | Amino acid | MAPRVTPLLAFSLLVLWTFPAPTLGGANDAEDCCLSVTQRPIPGNIVKAFRYLLNEDGCRVPAVVF |
| | sequence of | TTLRGYQLCAPPDQPWVDRIIRRLKKSSAKNKGNSTRRSPVS |
| | mouse CCL19 | |
| 19 | Nucleotide | ATGAGATCCAGTCCTGGCAACATGGAGAGGATTGTCATCTGTCTGATGGTCATCTTCTTGGGGA |
| | sequence of | CACTGGTCCACAAATCAAGCTCCCAAGGTCAAGATCGCCACATGATTAGAATGCGTCAACTTAT |
| | human IL-21 | AGATATTGTTGATCAGCTGAAAAATTATGTGAATGACTTGGTCCCTGAATTTCTGCCAGCTCCA |
| | | GAAGATGTAGAGACAAACTGTGAGTGGTCAGCTTTTTCCTGCTTTCAGAAGGCCCAACTAAAG |
| | | TCAGCAAATACAGGAAACAATGAAAGGATAATCAATGTATCAATTAAAAAGCTGAAGAGGAAA |
| | | CCACCTTCCACAAATGCAGGGAGAAGACAGAAACACAGACTAACATGCCCTTCATGTGATTCT |
| | | TATGAGAAAAAACCACCCAAAGAATTCCTAGAAAGATTCAAATCACTTCTCCAAAAGATGATT |
| | | CATCAGCATCTGTCCTCTAGAACACACGGAAGTGAAGATTCCTGA |
| 20 | Amino acid | MRSSPGNMERIVICLMVIFLGTLVHKSSSQGQDRHMIRMRQLIDIVDQLKNYVNDLVPEFLPAPED |
| | sequence of | VETNCEWSAFSCFQKAQLKSANTGNNERIINVSIKKLKRKPPSTNAGRRQKHRLTCPSCDSYEKKP |
| | human IL-21 | PKEFLERFKSLLQKMIHQHLSSRTHGSEDS |
| 21 | Nucleotide | ATGGCCCTGCTACTGGCCCTCAGCCTGCTGGTTCTCTGGACTTCCCCAGCCCCAACTCTGAGTG |
| | sequence of | GCACCAATGATGCTGAAGACTGCTGCCTGTCTGTGACCCAGAAACCCATCCCTGGGTACATCG |
| | human | TGAGGAACTTCCACTACCTTCTCATCAAGGATGGCTGCAGGGTGCCTGCTGTAGTGTTCACCA |
| | CCL 19 | CACTGAGGGGCCGCCAGCTCTGTGCACCCCCAGACCAGCCCTGGGTAGAACGCATCATCCAG |
| | | AGACTGCAGAGGACCTCAGCCAAGATGAAGCGCCGCAGCAGT |
| 22 | Amino acid | MALLLALSLLVLWTSPAPTLSGTNDAEDCCLSVTQKPIPGYIVRNEHYLLEKDGCRVPAVVFTTLRG |
| | sequence of | RQLCAPPDQPWVERIIQRLQRTSAKMKRRSS |
| | human | |
| | CCL 19 | |
| 23 | Amino acid | QVQLQESGPGLIKPSQTLSLTCTVSGGSISSGYNWHWIRQPPGKGLEWIGYIHYTGSTNYNPALRSR |
| | sequence of | VTISVDTSKNQFSLKLSSVTAADTAIYYCARIYNGNSFPYWGQGTTVTVSSGGGGSGGGGSGGGG |
| | Hu8E5-2I-28 | SDIVMTQSPDSLAVSLGERATINCKSSQSLFNSGNQKNYLTWYQQKPGQPPKLLIYWASTRESGVP |
| | Z CAR | DRFSGSGSGTDFTLTISSLQAEDVAVYYCQNAYSFPYTEGGGTKLEIKRTTTPAPRPPTPAPTIASQPL |
| | | SLRPEACRPAAGGAVHTRGLDFACDFWVLVVVGGVLACYSLLVTVAFIIFWVRSKRSRLLHSDYM |
| | | NMTPRRPGPTRKHYQPYAPPRDFAAYRSRVKFSRSADAPAYQQGQNQLYNELNLGRREEYDVLD |
| | | KRRGRDPEMGGKPQRRKNPQEGLYNELQKDKMAEAYSEIGMKGERRRGKGHDGLYQGLSTATK |
| | | DTYDALHMQALPPR |
| 24 | Amino acid | QVQLQESGPGLIKPSQTLSLTCTVSGGSISSGYNWHWIRQPPGKGLEWIGYIHYTGSTNYNPALRSR |
| | sequence of | VTISVDTSKNQFSLKLSSVTAADTAIYYCARIYNGNSFPYWGQGTTVTVSSGGGGSGGGGSGGGG |
| | Hu8E5-2I-BB | SDIVMTQSPDSLAVSLGERATINCKSSQSLFNSGNQKNYLTWYQQKPGQPPKLLIYWASTRESGVP |
| | Z CAR | DRFSGSGSGTDFTLTISSLQAEDVAVYYCQNAYSFPYTEGGGTKLEIKRTTTPAPRPPTPAPTIASQPL |
| | | SLRPEACRPAAGGAVHTRGLDFACDIYIWAPLAGTCGVLLLSLVITLYCKRGRKKLLYIFKQPFMRP |
| | | VQTTQEEDGCSCRFPEEEEGGCELRVKFSRSADAPAYQQGQNQLYNELNLGRREEYDVLDKRRG |
| | | RDPEMGGKPQRRKNPQEGLYNELQKDKMAEAYSEIGMKGERRRGKGHDGLYQGLSTATKDTYD |
| | | ALHMQALPPR |
| 25 | Amino acid | QVQLQESGPGLIKPSQTLSLTCTVSGGSISSGYNWHWIRQPPGKGLEWIGYIHYTGSTNYNPALRSR |
| | sequence of | VTISVDTSKNQFSLKLSSVTAADTAIYYCARIYNGNSFPYWGQGTTVTVSSGGGGSGGGGSGGGG |
| | Hu8E5-2I-28 | SDIVMTQSPDSLAVSLGERATINCKSSQSLFNSGNQKNYLTWYQQKPGQPPKLLIYWASTRESGVP |
| | BBZ CAR | DRFSGSGSGTDFTLTISSLQAEDVAVYYCQNAYSFPYTFGGGTKLEIKRTTTPAPRPPTPAPTIASQPL |
| | | SLRPEACRPAAGGAVHTRGLDFACDFWVLVVVGGVLACYSLLVTVAFIIFWVRSKRSRLLHSDYM |
| | | NMTPRRPGPTRKHYQPYAPPRDFAAYRSKRGRKKLLYIFKQPFMRPVQTTQEEDGCSCRFPEEEEG |
| | | GCELRVKFSRSADAPAYQQGQNQLYNELNLGRREEYDVLDKRRGRDPEMGGKPQRRKNPQEGLY |
| | | NELQKDKMAEAYSEIGMKGERRRGKGHDGLYQGLSTATKDTYDALHMQALPPR |
| 26 | Amino acid | QVQLQESGPGLIKPSQTLSLTCTVSGGSISSGYNWHWIRQPPGKGLEWIGYIHYTGSTNYNPALRSR |
| | sequence of | VTISVDTSKNQFSLKLSSVTAADTAIYYCARIYNGNSFPYWGQGTTVTVSSGGGGSGGGGSGGGG |
| | Hu8E5-2I-Z | SDIVMTQSPDSLAVSLGERATINCKSSQSLFNSGNQKNYLTWYQQKPGQPPKLLIYWASTRESGVP |
| | CAR | DRFSGSGSGTDFTLTISSLQAEDVAVYYCQNAYSFPYTFGGGTKLEIKRTTTPAPRPPTPAPTIASQPL |
| | | SLRPEACRPAAGGAVHTRGLDFACDIYIWAPLAGTCGVLLLSLVITRVKFSRSADAPAYQQGQNQL |
| | | YNELNLGRREEYDVLDKRRGRDPEMGGKPQRRKNPQEGLYNELQKDKMAEAYSEIGMKGERRR |
| | | GKGHDGLYQGLSTATKDTYDALHMQALPPR |
| 27 | Amino acid | MSTTTCQVVAFLLSILGLAGCIAATGMDMWSTQDLYDNPVTSVFQYEGLWRSCVRQSSGFTECRP |
| | sequence of | YFTILGLPAMLQAVRALMIVGIVLGAIGLLVSIFALKCIRIGSMEDSAKANMTLTSGIMFIVSGLCAI |
| | Claudin 18.1 | AGVSVFANMLVTNFWMSTANMYTGMGGMVQTVQTRYTFGAALFVGWVAGGLTLIGGVMMCIA |
| | | CRGLAPEETNYKAVSYHASGHSVAYKPGGFKASTGFGSNTKNKKIYDGGARTEDEVQSYPSKHD |
| | | YV |
| 28 | Amino acid | MAVTACQGLGFVVSLIGIAGIIAATCMDQWSTQDLYNNPVTAVFNYQGLWRSCVRESSGFTECRG |
| | sequence of | YFTLLGLPAMLQAVRALMIVGIVLGAIGLLVSIFALKCIRIGSMEDSAKANMTLTSGIMFIVSGLCAI |
| | Claudin 18.2 | AGVSVFANMLVTNFWMSTANMYTGMGGMVQTVQTRYTFGAALFVGWVAGGLTLIGGVMMCIA |
| | | CRGLAPEETNYKAVSYHASGHSVAYKPGGFKASTGFGSNTKNKKIYDGGARTEDEVQSYPSKHD |
| | | YV |
| 29 | Amino acid | MALPVTALLLPLALLLHAARP |
| | sequence of | |
| | human CD8 α | |
| | signal peptide | |
| 30 | Amino acid | TTTPAPRPPTPAPTIASQPLSLRPEACRPAAGGAVHTRGLDFACD |
| | sequence of | |
| | human CD8 | |
| | hinge | |
| 31 | Amino acid | FWVLVVVGGVLACYSLLVTVAFIIFWV |
| | sequence of | |
| | human CD28 | |
| | transmembrane | |
| | region | |
| 32 | Amino acid | RSKRSRLLHSDYMNMTPRRPGPTRKHYQPYAPPRDFAAYRS |
| | sequence of | |
| | human CD28 | |
| | intracellular | |
| | domain | |
| 33 | Amino acid | RVKFSRSADAPAYQQGQNQLYNELNLGRREEYDVLDKRRGRDPEMGGKPQRRKNPQEGLYNEL |
| | sequence of | QKDKMAEAYSEIGMKGERRRGKGHDGLYQGLSTATKDTYDALHMQALPPR |
| | intracellular | |
| | segment | |
| | CD3ξ of | |
| | human CD3 | |
| 34 | Amino acid | KRGRKKLLYIFKQPFMRPVQTTQEEDGCSCRFPEEEEGGCEL |
| | sequence of | |
| | human 4-1BB | |
| | intracellular | |
| | domain | |
| 35 | Amino acid | MAQSLALSLLILVLAFGIPRTQGSDGGAQDCCLKYSQRKIPAKVVRSYRKQEPSLGCSIPAILFLPRK |
| | sequence of | RSQAELCADPKELWVQQLMQHLDKTPSPQKPAQGCRKDRGASKTGKKGKGSKGCKRTERSQTP |
| | human | KGP |
| | CCL21 | |

All documents mentioned in the present invention are cited as references in this application, just as if each document is individually cited as a reference. In addition, it should be understood that after reading the above teaching content of the present invention, those skilled in the art can make various changes or modifications to the present invention, and these equivalent forms also fall within the scope defined by the appended claims of the present application.

Although the specific embodiments of the present invention are described above, those skilled in the art should understand that they are only examples, and various changes or modifications can be made to these embodiments without departing from the principle and essence of the present invention. Therefore, the protection scope of the present invention is defined by the appended claims.

1. A chemokine-expressing cell, wherein the cell expresses an exogenous IL-21R binding protein or an exogenous IL-21, and an exogenous chemokine;
preferably, the chemokine is an exogenous CCL19 or CCL21.

2. The cell of claim 1, wherein the cell expresses an exogenous IL-21 and an exogenous chemokine preferably, wherein the exogenous IL-21 is a wild-type IL-21 or a variant or truncated fragment of the wild-type IL-21, the variant or truncated fragment has the same or similar function as the wild-type IL-21;
more preferably, the exogenous IL-21 is a human or murine IL-21;
more preferably, the amino acid sequence of the exogenous IL-21 has at least 90% identity with the sequence shown in SEQ ID NO: 20.

3. (canceled)

4. The cell of any one of claim 1 or 2, wherein the exogenous IL-21R binding protein or the exogenous IL-21 is expressed constitutively or inductively;
preferably, the promoter for expressing the exogenous IL-21R binding protein or the exogenous IL-21 is an inducible promoter of an immune cell;
more preferably, the inducible promoter of an immune cell is the NFAT6 promoter.

5. The cell of claim 1, wherein the exogenous IL-21R binding protein can specifically bind to IL-21R and enhance IL-21R activity;
preferably, the IL-21R binding protein is selected from IL-21R antibodies.

6. The cell of claim 1, wherein the exogenous CCL19 is a wild-type CCL19 or a variant or truncated fragment of the wild-type CCL19, and the variant or truncated fragment has the same or similar function as the wild-type CCL19; preferably, the CCL19 is a human or murine CCL19;
more preferably, the amino acid sequence of the CCL19 has at least 90% identity with the sequence shown in SEQ ID NO: 22;
and/or, the exogenous CCL21 is a wild-type CCL21 or a variant or truncated fragment of the wild-type CCL21, and the variant or truncated fragment has the same or similar function as the wild-type CCL21; preferably, the CCL21 is a human or murine CCL21; more preferably, the amino acid sequence of the CCL21 has at least 90% identity with the sequence shown in SEQ ID NO: 35.

7. The cell of claim 1, wherein the exogenous chemokine is expressed constitutively or inductively;
preferably, the promoter used to express the chemokine is an inducible promoter of an immune cell;
more preferably, the inducible promoter of an immune cell is an NFAT6 promoter.

8. The cell of claim 1, wherein the cell further expresses an exogenous receptor that specifically binds to a target antigen;
preferably, the target antigen is tumor antigen or pathogen antigen;
more preferably, the target antigen is solid tumor-associated antigen;
more preferably, the solid tumor-associated antigen is selected from mesothelin, EGFR, EGFRvIII, GPC3, claudin18.2, claudin6 and IL13 R alpha.

9-11. (canceled)

12. The cell of claim 8, wherein the exogenous receptor has an antigen-binding domain, a transmembrane domain, and an intracellular domain, and the antigen-binding domain specifically binds to the target antigen;
preferably, the exogenous receptor is selected from the group consisting of: a chimeric antigen receptor (CAR), a modified T cell (antigen) receptor (TCR), a T cell fusion protein (TFP), a T cell antigen coupler (TAC), or a combination thereof.

13-14. (canceled)

15. The cell of claim 12, wherein the amino acid sequence of the antigen binding domain comprises a sequence that has at least 90% identity with the sequence shown in SEQ ID NO: 2;
preferably, the amino acid sequence of the exogenous receptor has at least 90% identity with the sequence shown in SEQ ID NO: 23, 24, 25, or 26.

16. (canceled)

17. The cell of claim 1, wherein the exogenous IL-21R binding protein or exogenous IL-21, and/or chemokine are expressed using a viral vector;
preferably, the viral vector comprises: a lentiviral vector, a retroviral vector or an adenoviral vector.

18. The cell of claim 8, wherein the exogenous receptor is expressed using a viral vector; preferably, the viral vector comprises: a lentiviral vector, a retroviral vector or an adenoviral vector.

19. The cell of claim 1, wherein the expression of an inhibitory immune checkpoint in the cell is down-regulated, and the inhibitory immune checkpoint is preferably PD-1, LAG-3 and/or TIM-3.

20. The cell of claim 1, wherein the cell is an immune effector cell; the immune effector cell is preferably selected from the group consisting of: a T cell, a B cell, a natural killer (NK) cell, and a natural killer T (NKT) cell, a mast cell, or a bone marrow-derived phagocyte, or a combination of at least two of them; the immune effector cell is more preferably a T cell, a B cell, or a NKT cell;
preferably, the cell is derived from an autologous cell or an allogeneic cell;
more preferably, the cell is an autologous T cell, an allogeneic T cell, or an allogeneic NK cell;
more preferably, the T cell is an autologous T cell.

21-23. (canceled)

24. A method for improving the viability of immune response cells, wherein the method comprises the co-expression of the following in immune response cells: a chimeric antigen receptor that specifically binds to a target antigen, an exogenous IL-21R binding protein or an exogenous IL-21, and an exogenous chemokine;
preferably, the chemokine is CCL_{19.}

25. A method for inhibiting tumors, inhibiting pathogens or strengthening subjects' immune tolerance, comprising giving the subject a pharmaceutical composition comprising the cell of any one of claims 1, 2, 5-8, 12, 15 and 17-20.

26-30. (canceled)

31. A method for inhibiting tumors, inhibiting pathogens or strengthening subjects' immune tolerance, comprising giving the subject a pharmaceutical composition comprising the cell of claim 4.
